SEQUENCE LISTING (282 sequences)

SEQ ID NO: 1-26 - skipped (26 entries)

SEQ ID NO: 27 - protein, 5 aa, synthetic construct
  REGION 1..5: note = non-recognition sequence
KLKLK

SEQ ID NO: 28 - protein, 5 aa, synthetic construct
  REGION 1..5: note = non-recognition sequence
KLQLK

SEQ ID NO: 29 - protein, 5 aa, synthetic construct
  REGION 1..5: note = non-recognition sequence
QLKLK

SEQ ID NO: 30 - protein, 5 aa, synthetic construct
  REGION 1..5: note = non-recognition sequence
KQKLK

SEQ ID NO: 31 - skipped

SEQ ID NO: 32 - protein, 5 aa, synthetic construct
  REGION 1..5: note = non-recognition sequence
ELKLK

SEQ ID NO: 33 - protein, 5 aa, synthetic construct
  REGION 1..5: note = recognition sequence
MVVSW

SEQ ID NO: 34 - protein, 5 aa, synthetic construct
  REGION 1..5: note = Recognition sequence
MVVSA

SEQ ID NO: 35 - protein, 5 aa, synthetic construct
  REGION 1..5: note = Recognition sequence
MVVAW

SEQ ID NO: 36 - protein, 5 aa, synthetic construct
  REGION 1..5: note = Recognition sequence
MVASW

SEQ ID NO: 37 - protein, 5 aa, synthetic construct
  REGION 1..5: note = Recognition sequence
MAVSW

SEQ ID NO: 38 - protein, 5 aa, synthetic construct
  REGION 1..5: note = Recognition sequence
AVVSW

SEQ ID NO: 39 - protein, 5 aa, synthetic construct
  REGION 1..5: note = Recognition sequence
  SITE 1: note = norleucine
XVVSW

SEQ ID NO: 40 - protein, 5 aa, synthetic construct
  REGION 1..5: note = Recognition sequence
  SITE 1: note = norleucine
XVVYW

SEQ ID NO: 41 - protein, 5 aa, synthetic construct
  REGION 1..5: note = Recognition sequence
  SITE 1: note = norleucine
XVVAW

SEQ ID NO: 42 - protein, 5 aa, synthetic construct
  REGION 1..5: note = Recognition sequence
AVVAW

SEQ ID NO: 43 - protein, 5 aa, synthetic construct
  REGION 1..5: note = Recognition sequence
  SITE 1: note = norleucine
XAVAW

SEQ ID NO: 44 - protein, 5 aa, synthetic construct
  REGION 1..5: note = Recognition sequence
  SITE 1: note = norleucine
XVAAW

SEQ ID NO: 45 - protein, 5 aa, synthetic construct
  REGION 1..5: note = Recognition sequence
  SITE 1: note = norleucine
XVLAW

SEQ ID NO: 46 - protein, 5 aa, synthetic construct
  REGION 1..5: note = Recognition sequence
  SITE 1: note = norleucine
XVIAW

SEQ ID NO: 47 - protein, 5 aa, synthetic construct
  REGION 1..5: note = Recognition sequence
  SITE 1: note = norleucine
XVFAW

SEQ ID NO: 48 - protein, 5 aa, synthetic construct
  REGION 1..5: note = Recognition sequence
WSVVW

SEQ ID NO: 49 - skipped

SEQ ID NO: 50 - protein, 5 aa, synthetic construct
  REGION 1..5: note = Recognition sequence
WAVAW

SEQ ID NO: 51 - protein, 5 aa, synthetic construct
  REGION 1..5: note = Recognition sequence
WAVAA

SEQ ID NO: 52 - protein, 5 aa, synthetic construct
  REGION 1..5: note = Recognition sequence
WAVAM

SEQ ID NO: 53 - protein, 5 aa, synthetic construct
  REGION 1..5: note = Recognition sequence
  SITE 5: note = norleucine
WAVAX

SEQ ID NO: 54 - protein, 5 aa, synthetic construct
  REGION 1..5: note = Recognition sequence
  SITE 5: note = norleucine
WAVVX

SEQ ID NO: 55 - protein, 5 aa, synthetic construct
  REGION 1..5: note = Recognition sequence
  SITE 5: note = norleucine
WAVSX

SEQ ID NO: 56 - protein, 5 aa, synthetic construct
  REGION 1..5: note = Recognition sequence
WAAAW

SEQ ID NO: 57 - protein, 5 aa, synthetic construct
  REGION 1..5: note = Recognition sequence
WAAAA

SEQ ID NO: 58 - protein, 5 aa, synthetic construct
  REGION 1..5: note = Recognition sequence
WAAAM

SEQ ID NO: 59 - protein, 5 aa, synthetic construct
  REGION 1..5: note = Recognition sequence
  SITE 5: note = norleucine
WAAAX

SEQ ID NO: 60 - protein, 5 aa, synthetic construct
  REGION 1..5: note = Recognition sequence
WAAVW

SEQ ID NO: 61 - protein, 5 aa, synthetic construct
  REGION 1..5: note = Recognition sequence
WAAVA

SEQ ID NO: 62 - protein, 5 aa, synthetic construct
  REGION 1..5: note = Recognition sequence
WAAVM

SEQ ID NO: 63 - protein, 5 aa, synthetic construct
  REGION 1..5: note = Recognition sequence
  SITE 5: note = norleucine
WAAVX

SEQ ID NO: 64 - protein, 5 aa, synthetic construct
  REGION 1..5: note = Recognition sequence
  SITE 5: note = norleucine
WAASX

SEQ ID NO: 65 - protein, 5 aa, synthetic construct
  REGION 1..5: note = Recognition sequence
WVVAW

SEQ ID NO: 66 - protein, 5 aa, synthetic construct
  REGION 1..5: note = Recognition sequence
WVVAA

SEQ ID NO: 67 - protein, 5 aa, synthetic construct
  REGION 1..5: note = Recognition sequence
WVVAM

SEQ ID NO: 68 - protein, 5 aa, synthetic construct
  REGION 1..5: note = Recognition sequence
  SITE 5: note = norleucine
WVVAX

SEQ ID NO: 69 - protein, 5 aa, synthetic construct
  REGION 1..5: note = Recognition sequence
WVVVW

SEQ ID NO: 70 - protein, 5 aa, synthetic construct
  REGION 1..5: note = Recognition sequence
WVVVA

SEQ ID NO: 71 - protein, 5 aa, synthetic construct
  REGION 1..5: note = Recognition sequence
WVVVM

SEQ ID NO: 72 - protein, 5 aa, synthetic construct
  REGION 1..5: note = Recognition sequence
  SITE 5: note = norleucine
WVVVX

SEQ ID NO: 73 - protein, 5 aa, synthetic construct
  REGION 1..5: note = Recognition sequence
  SITE 5: note = norleucine
WVVSX

SEQ ID NO: 74 - protein, 5 aa, synthetic construct
  REGION 1..5: note = Recognition sequence
  SITE 5: note = norleucine
WVAAX

SEQ ID NO: 75 - protein, 5 aa, synthetic construct
  REGION 1..5: note = Recognition sequence
WVAVW

SEQ ID NO: 76 - protein, 5 aa, synthetic construct
  REGION 1..5: note = Recognition sequence
WVAVA

SEQ ID NO: 77 - protein, 5 aa, synthetic construct
  REGION 1..5: note = Recognition sequence
WVAVM

SEQ ID NO: 78 - protein, 5 aa, synthetic construct
  REGION 1..5: note = Recognition sequence
  SITE 5: note = norleucine
WVAVX

SEQ ID NO: 79 - protein, 5 aa, synthetic construct
  REGION 1..5: note = Recognition sequence
  SITE 5: note = norleucine
WVASX

SEQ ID NO: 80 - protein, 5 aa, synthetic construct
  REGION 1..5: note = Recognition sequence
WSVAW

SEQ ID NO: 81 - protein, 5 aa, synthetic construct
  REGION 1..5: note = Recognition sequence
WSVAA

SEQ ID NO: 82 - protein, 5 aa, synthetic construct
  REGION 1..5: note = Recognition sequence
WSVAM

SEQ ID NO: 83 - protein, 5 aa, synthetic construct
  REGION 1..5: note = Recognition sequence
  SITE 5: note = norleucine
WSVAX

SEQ ID NO: 84 - protein, 5 aa, synthetic construct
  REGION 1..5: note = Recognition sequence
WSVVA

SEQ ID NO: 85 - protein, 5 aa, synthetic construct
  REGION 1..5: note = Recognition sequence
WSVVM

SEQ ID NO: 86 - protein, 5 aa, synthetic construct
  REGION 1..5: note = Recognition sequence
  SITE 5: note = norleucine
WSVVX

SEQ ID NO: 87 - protein, 5 aa, synthetic construct
  REGION 1..5: note = Recognition sequence
WSVSW

SEQ ID NO: 88 - protein, 5 aa, synthetic construct
  REGION 1..5: note = Recognition sequence
WSVSA

SEQ ID NO: 89 - protein, 5 aa, synthetic construct
  REGION 1..5: note = Recognition sequence
WSVSM

SEQ ID NO: 90 - protein, 5 aa, synthetic construct
  REGION 1..5: note = Recognition sequence
  SITE 5: note = norleucine
WSVSX

SEQ ID NO: 91 - protein, 5 aa, synthetic construct
  REGION 1..5: note = Recognition sequence
WSAAW

SEQ ID NO: 92 - protein, 5 aa, synthetic construct
  REGION 1..5: note = Recognition sequence
WSAAA

SEQ ID NO: 93 - protein, 5 aa, synthetic construct
  REGION 1..5: note = Recognition sequence
WSAAM

SEQ ID NO: 94 - protein, 5 aa, synthetic construct
  REGION 1..5: note = Recognition sequence
  SITE 5: note = norleucine
WSAAX

SEQ ID NO: 95 - protein, 5 aa, synthetic construct
  REGION 1..5: note = Recognition sequence
WSAVW

SEQ ID NO: 96 - protein, 5 aa, synthetic construct
  REGION 1..5: note = Recognition sequence
WSAVA

SEQ ID NO: 97 - protein, 5 aa, synthetic construct
  REGION 1..5: note = Recognition sequence
WSAVM

SEQ ID NO: 98 - protein, 5 aa, synthetic construct
  REGION 1..5: note = Recognition sequence
  SITE 5: note = norleucine
WSAVX

SEQ ID NO: 99 - protein, 5 aa, synthetic construct
  REGION 1..5: note = Recognition sequence
WSASW

SEQ ID NO: 100 - protein, 5 aa, synthetic construct
  REGION 1..5: note = Recognition sequence
WSASA

SEQ ID NO: 101 - protein, 5 aa, synthetic construct
  REGION 1..5: note = Recognition sequence
WSASM

SEQ ID NO: 102 - protein, 5 aa, synthetic construct
  REGION 1..5: note = Recognition sequence
  SITE 5: note = norleucine
WSASX

SEQ ID NO: 103 - protein, 5 aa, synthetic construct
  REGION 1..5: note = Recognition sequence
WYVAW

SEQ ID NO: 104 - protein, 5 aa, synthetic construct
  REGION 1..5: note = Recognition sequence
WYVAA

SEQ ID NO: 105 - protein, 5 aa, synthetic construct
  REGION 1..5: note = Recognition sequence
WYVAM

SEQ ID NO: 106 - protein, 5 aa, synthetic construct
  REGION 1..5: note = Recognition sequence
  SITE 5: note = norleucine
WYVAX

SEQ ID NO: 107 - protein, 5 aa, synthetic construct
  REGION 1..5: note = Recognition sequence
WYVVW

SEQ ID NO: 108 - protein, 5 aa, synthetic construct
  REGION 1..5: note = Recognition sequence
WYVVA

SEQ ID NO: 109 - protein, 5 aa, synthetic construct
  REGION 1..5: note = Recognition sequence
WYVVM

SEQ ID NO: 110 - protein, 5 aa, synthetic construct
  REGION 1..5: note = Recognition sequence
  SITE 5: note = norleucine
WYVVX

SEQ ID NO: 111 - protein, 5 aa, synthetic construct
  REGION 1..5: note = Recognition sequence
WYVSW

SEQ ID NO: 112 - protein, 5 aa, synthetic construct
  REGION 1..5: note = Recognition sequence
WYVSA

SEQ ID NO: 113 - protein, 5 aa, synthetic construct
  REGION 1..5: note = Recognition sequence
WYVSM

SEQ ID NO: 114 - protein, 5 aa, synthetic construct
  REGION 1..5: note = Recognition sequence
  SITE 5: note = norleucine
WYVSX

SEQ ID NO: 115 - protein, 5 aa, synthetic construct
  REGION 1..5: note = Recognition sequence
WYAAW

SEQ ID NO: 116 - protein, 5 aa, synthetic construct
  REGION 1..5: note = Recognition sequence
WYAAA

SEQ ID NO: 117 - protein, 5 aa, synthetic construct
  REGION 1..5: note = Recognition sequence
WYAAM

SEQ ID NO: 118 - protein, 5 aa, synthetic construct
  REGION 1..5: note = Recognition sequence
  SITE 5: note = norleucine
WYAAX

SEQ ID NO: 119 - protein, 5 aa, synthetic construct
  REGION 1..5: note = Recognition sequence
WYAAW

SEQ ID NO: 120 - protein, 5 aa, synthetic construct
  REGION 1..5: note = Recognition sequence
WYAVA

SEQ ID NO: 121 - protein, 5 aa, synthetic construct
  REGION 1..5: note = Recognition sequence
WYAVM

SEQ ID NO: 122 - protein, 5 aa, synthetic construct
  REGION 1..5: note = Recognition sequence
  SITE 5: note = norleucine
WYAVX

SEQ ID NO: 123 - protein, 5 aa, synthetic construct
  REGION 1..5: note = Recognition sequence
WYASW

SEQ ID NO: 124 - protein, 5 aa, synthetic construct
  REGION 1..5: note = Recognition sequence
WYASA

SEQ ID NO: 125 - protein, 5 aa, synthetic construct
  REGION 1..5: note = Recognition sequence
WYASM

SEQ ID NO: 126 - protein, 5 aa, synthetic construct
  REGION 1..5: note = Recognition sequence
  SITE 5: note = norleucine
WYASX

SEQ ID NO: 127 - protein, 5 aa, synthetic construct
  REGION 1..5: note = Recognition sequence
AAVAA

SEQ ID NO: 128 - protein, 5 aa, synthetic construct
  REGION 1..5: note = Recognition sequence
AAVAM

SEQ ID NO: 129 - protein, 5 aa, synthetic construct
  REGION 1..5: note = Recognition sequence
  SITE 5: note = norleucine
AAVAX

SEQ ID NO: 130 - protein, 5 aa, synthetic construct
  REGION 1..5: note = Recognition sequence
  SITE 5: note = norleucine
AAVVX

SEQ ID NO: 131 - protein, 5 aa, synthetic construct
  REGION 1..5: note = Recognition sequence
  SITE 5: note = norleucine
AAVAX

SEQ ID NO: 132 - protein, 5 aa, synthetic construct
  REGION 1..5: note = Recognition sequence
AAAAA

SEQ ID NO: 133 - protein, 5 aa, synthetic construct
  REGION 1..5: note = Recognition sequence
AAAAM

SEQ ID NO: 134 - protein, 5 aa, synthetic construct
  REGION 1..5: note = Recognition sequence
  SITE 5: note = norleucine
AAAAX

SEQ ID NO: 135 - protein, 5 aa, synthetic construct
  REGION 1..5: note = Recognition sequence
AAAVW

SEQ ID NO: 136 - protein, 5 aa, synthetic construct
  REGION 1..5: note = Recognition sequence
AAAVA

SEQ ID NO: 137 - protein, 5 aa, synthetic construct
  REGION 1..5: note = Recognition sequence
AAAVM

SEQ ID NO: 138 - protein, 5 aa, synthetic construct
  REGION 1..5: note = Recognition sequence
  SITE 5: note = norleucine
AAAVX

SEQ ID NO: 139 - protein, 5 aa, synthetic construct
  REGION 1..5: note = Recognition sequence
AAASM

SEQ ID NO: 140 - protein, 5 aa, synthetic construct
  REGION 1..5: note = Recognition sequence
  SITE 5: note = norleucine
AAASX

SEQ ID NO: 141 - protein, 5 aa, synthetic construct
  REGION 1..5: note = Recognition sequence
AVVAW

SEQ ID NO: 142 - protein, 5 aa, synthetic construct
  REGION 1..5: note = Recognition sequence
AVVAA

SEQ ID NO: 143 - protein, 5 aa, synthetic construct
  REGION 1..5: note = Recognition sequence
AVVAM

SEQ ID NO: 144 - protein, 5 aa, synthetic construct
  REGION 1..5: note = Recognition sequence
  SITE 5: note = norleucine
AVVAX

SEQ ID NO: 145 - protein, 5 aa, synthetic construct
  REGION 1..5: note = Recognition sequence
AVVVA

SEQ ID NO: 146 - protein, 5 aa, synthetic construct
  REGION 1..5: note = Recognition sequence
AVVVM

SEQ ID NO: 147 - protein, 5 aa, synthetic construct
  REGION 1..5: note = Recognition sequence
  SITE 5: note = norleucine
AVVVX

SEQ ID NO: 148 - protein, 5 aa, synthetic construct
  REGION 1..5: note = Recognition sequence
  SITE 5: note = norleucine
AVVSX

SEQ ID NO: 149 - protein, 5 aa, synthetic construct
  REGION 1..5: note = Recognition sequence
AVAAM

SEQ ID NO: 150 - protein, 5 aa, synthetic construct
  REGION 1..5: note = Recognition sequence
  SITE 5: note = norleucine
AVAAX

SEQ ID NO: 151 - protein, 5 aa, synthetic construct
  REGION 1..5: note = Recognition sequence
AVAVA

SEQ ID NO: 152 - protein, 5 aa, synthetic construct
  REGION 1..5: note = Recognition sequence
AVAVM

SEQ ID NO: 153 - protein, 5 aa, synthetic construct
  REGION 1..5: note = Recognition sequence
  SITE 5: note = norleucine
AVAVX

SEQ ID NO: 154 - protein, 5 aa, synthetic construct
  REGION 1..5: note = Recognition sequence
  SITE 5: note = norleucine
AVASX

SEQ ID NO: 155 - protein, 5 aa, synthetic construct
  REGION 1..5: note = Recognition sequence
ASVAW

SEQ ID NO: 156 - protein, 5 aa, synthetic construct
  REGION 1..5: note = Recognition sequence
ASVAA

SEQ ID NO: 157 - protein, 5 aa, synthetic construct
  REGION 1..5: note = Recognition sequence
ASVAM

SEQ ID NO: 158 - protein, 5 aa, synthetic construct
  REGION 1..5: note = Recognition sequence
  SITE 5: note = norleucine
ASVAX

SEQ ID NO: 159 - protein, 5 aa, synthetic construct
  REGION 1..5: note = Recognition sequence
ASVVW

SEQ ID NO: 160 - protein, 5 aa, synthetic construct
  REGION 1..5: note = Recognition sequence
ASVVA

SEQ ID NO: 161 - protein, 5 aa, synthetic construct
  REGION 1..5: note = Recognition sequence
ASVVM

SEQ ID NO: 162 - protein, 5 aa, synthetic construct
  REGION 1..5: note = Recognition sequence
  SITE 5: note = norleucine
ASVVX

SEQ ID NO: 163 - protein, 5 aa, synthetic construct
  REGION 1..5: note = Recognition sequence
ASVSA

SEQ ID NO: 164 - protein, 5 aa, synthetic construct
  REGION 1..5: note = Recognition sequence
ASVSM

SEQ ID NO: 165 - protein, 5 aa, synthetic construct
  REGION 1..5: note = Recognition sequence
  SITE 5: note = norleucine
ASVSX

SEQ ID NO: 166 - protein, 5 aa, synthetic construct
  REGION 1..5: note = Recognition sequence
ASAAW

SEQ ID NO: 167 - protein, 5 aa, synthetic construct
  REGION 1..5: note = Recognition sequence
ASAAA

SEQ ID NO: 168 - protein, 5 aa, synthetic construct
  REGION 1..5: note = Recognition sequence
ASAAM

SEQ ID NO: 169 - protein, 5 aa, synthetic construct
  REGION 1..5: note = Recognition sequence
  SITE 5: note = norleucine
ASAAX

SEQ ID NO: 170 - protein, 5 aa, synthetic construct
  REGION 1..5: note = Recognition sequence
ASAVW

SEQ ID NO: 171 - protein, 5 aa, synthetic construct
  REGION 1..5: note = Recognition sequence
ASAVA

SEQ ID NO: 172 - protein, 5 aa, synthetic construct
  REGION 1..5: note = Recognition sequence
ASAVM

SEQ ID NO: 173 - protein, 5 aa, synthetic construct
  REGION 1..5: note = Recognition sequence
  SITE 5: note = norleucine
ASAVX

SEQ ID NO: 174 - protein, 5 aa, synthetic construct
  REGION 1..5: note = Recognition sequence
ASASA

SEQ ID NO: 175 - protein, 5 aa, synthetic construct
  REGION 1..5: note = Recognition sequence
ASASM

SEQ ID NO: 176 - protein, 5 aa, synthetic construct
  REGION 1..5: note = Recognition sequence
  SITE 5: note = norleucine
ASASX

SEQ ID NO: 177 - protein, 5 aa, synthetic construct
  REGION 1..5: note = Recognition sequence
AYVAW

SEQ ID NO: 178 - protein, 5 aa, synthetic construct
  REGION 1..5: note = Recognition sequence
AYVAA

SEQ ID NO: 179 - protein, 5 aa, synthetic construct
  REGION 1..5: note = Recognition sequence
AYVAM

SEQ ID NO: 180 - protein, 5 aa, synthetic construct
  REGION 1..5: note = Recognition sequence
  SITE 5: note = norleucine
AYVAX

SEQ ID NO: 181 - protein, 5 aa, synthetic construct
  REGION 1..5: note = Recognition sequence
AYVVW

SEQ ID NO: 182 - protein, 5 aa, synthetic construct
  REGION 1..5: note = Recognition sequence
AYVVA

SEQ ID NO: 183 - protein, 5 aa, synthetic construct
  REGION 1..5: note = Recognition sequence
AYVVM

SEQ ID NO: 184 - protein, 5 aa, synthetic construct
  REGION 1..5: note = Recognition sequence
  SITE 5: note = norleucine
AYVVX

SEQ ID NO: 185 - protein, 5 aa, synthetic construct
  REGION 1..5: note = Recognition sequence
AYVSW

SEQ ID NO: 186 - protein, 5 aa, synthetic construct
  REGION 1..5: note = Recognition sequence
AYVSA

SEQ ID NO: 187 - protein, 5 aa, synthetic construct
  REGION 1..5: note = Recognition sequence
AYVSM

SEQ ID NO: 188 - protein, 5 aa, synthetic construct
  REGION 1..5: note = Recognition sequence
  SITE 5: note = norleucine
AYVSX

SEQ ID NO: 189 - protein, 5 aa, synthetic construct
  REGION 1..5: note = Recognition sequence
AYAAW

SEQ ID NO: 190 - protein, 5 aa, synthetic construct
  REGION 1..5: note = Recognition sequence
AYAAA

SEQ ID NO: 191 - protein, 5 aa, synthetic construct
  REGION 1..5: note = Recognition sequence
AYAAM

SEQ ID NO: 192 - protein, 5 aa, synthetic construct
  REGION 1..5: note = Recognition sequence
  SITE 5: note = norleucine
AYAAX

SEQ ID NO: 193 - protein, 5 aa, synthetic construct
  REGION 1..5: note = Recognition sequence
AYAVW

SEQ ID NO: 194 - protein, 5 aa, synthetic construct
  REGION 1..5: note = Recognition sequence
AYAVA

SEQ ID NO: 195 - protein, 5 aa, synthetic construct
  REGION 1..5: note = Recognition sequence
AYAVM

SEQ ID NO: 196 - protein, 5 aa, synthetic construct
  REGION 1..5: note = Recognition sequence
  SITE 5: note = norleucine
AYAVX

SEQ ID NO: 197 - protein, 5 aa, synthetic construct
  REGION 1..5: note = Recognition sequence
AYASW

SEQ ID NO: 198 - protein, 5 aa, synthetic construct
  REGION 1..5: note = Recognition sequence
AYASA

SEQ ID NO: 199 - protein, 5 aa, synthetic construct
  REGION 1..5: note = Recognition sequence
AYASM

SEQ ID NO: 200 - protein, 5 aa, synthetic construct
  REGION 1..5: note = Recognition sequence
  SITE 5: note = norleucine
AYASX

SEQ ID NO: 201 - protein, 5 aa, synthetic construct
  REGION 1..5: note = Recognition sequence
MAVAA

SEQ ID NO: 202 - protein, 5 aa, synthetic construct
  REGION 1..5: note = Recognition sequence
MAVAM

SEQ ID NO: 203 - protein, 5 aa, synthetic construct
  REGION 1..5: note = Recognition sequence
  SITE 5: note = norleucine
MAVAX

SEQ ID NO: 204 - protein, 5 aa, synthetic construct
  REGION 1..5: note = Recognition sequence
  SITE 5: note = norleucine
MAVVX

SEQ ID NO: 205 - protein, 5 aa, synthetic construct
  REGION 1..5: note = Recognition sequence
  SITE 5: note = norleucine
MAVSX

SEQ ID NO: 206 - protein, 5 aa, synthetic construct
  REGION 1..5: note = Recognition sequence
MAAAA

SEQ ID NO: 207 - protein, 5 aa, synthetic construct
  REGION 1..5: note = Recognition sequence
MAAAM

SEQ ID NO: 208 - protein, 5 aa, synthetic construct
  REGION 1..5: note = Recognition sequence
  SITE 5: note = norleucine
MAAAX

SEQ ID NO: 209 - protein, 5 aa, synthetic construct
  REGION 1..5: note = Recognition sequence
MAAVW

SEQ ID NO: 210 - protein, 5 aa, synthetic construct
  REGION 1..5: note = Recognition sequence
MAAVA

SEQ ID NO: 211 - protein, 5 aa, synthetic construct
  REGION 1..5: note = Recognition sequence
MAAVM

SEQ ID NO: 212 - protein, 5 aa, synthetic construct
  REGION 1..5: note = Recognition sequence
  SITE 5: note = norleucine
MAAVX

SEQ ID NO: 213 - protein, 5 aa, synthetic construct
  REGION 1..5: note = Recognition sequence
  SITE 5: note = norleucine
MAASX

SEQ ID NO: 214 - protein, 5 aa, synthetic construct
  REGION 1..5: note = Recognition sequence
MVVAW

SEQ ID NO: 215 - protein, 5 aa, synthetic construct
  REGION 1..5: note = Recognition sequence
MVVAA

SEQ ID NO: 216 - protein, 5 aa, synthetic construct
  REGION 1..5: note = Recognition sequence
MVVAM

SEQ ID NO: 217 - protein, 5 aa, synthetic construct
  REGION 1..5: note = Recognition sequence
  SITE 5: note = norleucine
MVVAX

SEQ ID NO: 218 - protein, 5 aa, synthetic construct
  REGION 1..5: note = Recognition sequence
MVVVM

SEQ ID NO: 219 - protein, 5 aa, synthetic construct
  REGION 1..5: note = Recognition sequence
  SITE 5: note = norleucine
MVVVX

SEQ ID NO: 220 - protein, 5 aa, synthetic construct
  REGION 1..5: note = Recognition sequence
  SITE 5: note = norleucine
MVVSX

SEQ ID NO: 221 - protein, 5 aa, synthetic construct
  REGION 1..5: note = Recognition sequence
MVAAM

SEQ ID NO: 222 - protein, 5 aa, synthetic construct
  REGION 1..5: note = Recognition sequence
  SITE 5: note = norleucine
MVAAX

SEQ ID NO: 223 - protein, 5 aa, synthetic construct
  REGION 1..5: note = Recognition sequence
MVAVM

SEQ ID NO: 224 - protein, 5 aa, synthetic construct
  REGION 1..5: note = Recognition sequence
  SITE 5: note = norleucine
MVAVX

SEQ ID NO: 225 - protein, 5 aa, synthetic construct
  REGION 1..5: note = Recognition sequence
  SITE 5: note = norleucine
MVASX

SEQ ID NO: 226 - protein, 5 aa, synthetic construct
  REGION 1..5: note = Recognition sequence
MSVAW

SEQ ID NO: 227 - protein, 5 aa, synthetic construct
  REGION 1..5: note = Recognition sequence
MSVAA

SEQ ID NO: 228 - protein, 5 aa, synthetic construct
  REGION 1..5: note = Recognition sequence
MSVAM

SEQ ID NO: 229 - protein, 5 aa, synthetic construct
  REGION 1..5: note = Recognition sequence
  SITE 5: note = norleucine
MSVAX

SEQ ID NO: 230 - protein, 5 aa, synthetic construct
  REGION 1..5: note = Recognition sequence
MSVVW

SEQ ID NO: 231 - protein, 5 aa, synthetic construct
  REGION 1..5: note = Recognition sequence
MSVVA

SEQ ID NO: 232 - protein, 5 aa, synthetic construct
  REGION 1..5: note = Recognition sequence
MSVVM

SEQ ID NO: 233 - protein, 5 aa, synthetic construct
  REGION 1..5: note = Recognition sequence
  SITE 5: note = norleucine
MSVVX

SEQ ID NO: 234 - protein, 5 aa, synthetic construct
  REGION 1..5: note = Recognition sequence
MSVSM

SEQ ID NO: 235 - protein, 5 aa, synthetic construct
  REGION 1..5: note = Recognition sequence
  SITE 5: note = norleucine
MSVSX

SEQ ID NO: 236 - protein, 5 aa, synthetic construct
  REGION 1..5: note = Recognition sequence
MSAAW

SEQ ID NO: 237 - protein, 5 aa, synthetic construct
  REGION 1..5: note = Recognition sequence
MSAAA

SEQ ID NO: 238 - protein, 5 aa, synthetic construct
  REGION 1..5: note = Recognition sequence
MSAAM

SEQ ID NO: 239 - protein, 5 aa, synthetic construct
  REGION 1..5: note = Recognition sequence
  SITE 5: note = norleucine
MSAAX

SEQ ID NO: 240 - protein, 5 aa, synthetic construct
  REGION 1..5: note = Recognition sequence
MSAVW

SEQ ID NO: 241 - protein, 5 aa, synthetic construct
  REGION 1..5: note = Recognition sequence
MSAVA

SEQ ID NO: 242 - protein, 5 aa, synthetic construct
  REGION 1..5: note = Recognition sequence
MSAVM

SEQ ID NO: 243 - protein, 5 aa, synthetic construct
  REGION 1..5: note = Recognition sequence
  SITE 5: note = norleucine
MSAVX

SEQ ID NO: 244 - protein, 5 aa, synthetic construct
  REGION 1..5: note = Recognition sequence
MSASM

SEQ ID NO: 245 - protein, 5 aa, synthetic construct
  REGION 1..5: note = Recognition sequence
  SITE 5: note = norleucine
MSASX

SEQ ID NO: 246 - protein, 5 aa, synthetic construct
  REGION 1..5: note = Recognition sequence
MYVAW

SEQ ID NO: 247 - protein, 5 aa, synthetic construct
  REGION 1..5: note = Recognition sequence
MYVAA

SEQ ID NO: 248 - protein, 5 aa, synthetic construct
  REGION 1..5: note = Recognition sequence
MYVAM

SEQ ID NO: 249 - protein, 5 aa, synthetic construct
  REGION 1..5: note = Recognition sequence
  SITE 5: note = norleucine
MYVAX

SEQ ID NO: 250 - protein, 5 aa, synthetic construct
  REGION 1..5: note = Recognition sequence
MYVVW

SEQ ID NO: 251 - protein, 5 aa, synthetic construct
  REGION 1..5: note = Recognition sequence
MYVVA

SEQ ID NO: 252 - protein, 5 aa, synthetic construct
  REGION 1..5: note = Recognition sequence
MYVVM

SEQ ID NO: 253 - protein, 5 aa, synthetic construct
  REGION 1..5: note = Recognition sequence
  SITE 5: note = norleucine
MYVVX

SEQ ID NO: 254 - protein, 5 aa, synthetic construct
  REGION 1..5: note = Recognition sequence
MYVSW

SEQ ID NO: 255 - protein, 5 aa, synthetic construct
  REGION 1..5: note = Recognition sequence
MYVSA

SEQ ID NO: 256 - protein, 5 aa, synthetic construct
  REGION 1..5: note = Recognition sequence
MYVSM

SEQ ID NO: 257 - protein, 5 aa, synthetic construct
  REGION 1..5: note = Recognition sequence
  SITE 5: note = norleucine
MYVSX

SEQ ID NO: 258 - protein, 5 aa, synthetic construct
  REGION 1..5: note = Recognition sequence
MYAAW

SEQ ID NO: 259 - protein, 5 aa, synthetic construct
  REGION 1..5: note = Recognition sequence
MYAAA

SEQ ID NO: 260 - protein, 5 aa, synthetic construct
  REGION 1..5: note = Recognition sequence
MYAAM

SEQ ID NO: 261 - protein, 5 aa, synthetic construct
  REGION 1..5: note = Recognition sequence
  SITE 5: note = norleucine
MYAAX

SEQ ID NO: 262 - protein, 5 aa, synthetic construct
  REGION 1..5: note = Recognition sequence
MYAVW

SEQ ID NO: 263 - protein, 5 aa, synthetic construct
  REGION 1..5: note = Recognition sequence
MYAVA

SEQ ID NO: 264 - protein, 5 aa, synthetic construct
  REGION 1..5: note = Recognition sequence
MYAVM

SEQ ID NO: 265 - protein, 5 aa, synthetic construct
  REGION 1..5: note = Recognition sequence
  SITE 5: note = norleucine
MYAVX

SEQ ID NO: 266 - protein, 5 aa, synthetic construct
  REGION 1..5: note = Recognition sequence
MYASW

SEQ ID NO: 267 - protein, 5 aa, synthetic construct
  REGION 1..5: note = Recognition sequence
MYASA

SEQ ID NO: 268 - protein, 5 aa, synthetic construct
  REGION 1..5: note = Recognition sequence
MYASM

SEQ ID NO: 269 - protein, 5 aa, synthetic construct
  REGION 1..5: note = Recognition sequence
  SITE 5: note = norleucine
MYASX

SEQ ID NO: 270-280 - skipped (11 entries)

SEQ ID NO: 281 - protein, 5 aa, synthetic construct
  REGION 1..5: note = Recognition sequence
KLKLQ

SEQ ID NO: 282 - protein, 5 aa, synthetic construct
  REGION 1..5: note = Recognition sequence
KLKQK

(19) United States
(12) Patent Application Publication
(10) Pub. No.: US 2025/0136642 A1
(43) Pub. Date: May 1, 2025

(54) CYCLIC PEPTIDE COMPOUNDS AND METHODS OF USE THEREOF

(71) Applicant: Modulation Therapeutics, Inc., Morgantown, WV (US)

(72) Inventors: Mark MCLAUGHLIN, Morgantown, WV (US); Lori HAZLEHURST, Morgantown, WV (US)

(21) Appl. No.: 18/763,815

(22) Filed: Jul. 3, 2024

Related U.S. Application Data
(63) Continuation of application No. 16/972,558, filed on Dec. 4, 2020, now Pat. No. 12,060,438.
(60) Provisional application No. 62/680,904, filed on Jun. 5, 2018.

(51) Int. Cl. C07K 7/64; A61K 38/00

(52) U.S. Cl. CPC C07K 7/64; A61K 38/00

(57) ABSTRACT

Embodiments of the present disclosure generally relate to cyclic peptidomimetic compounds and methods for making the same. Other embodiments relate to a method of treating a disease by administering a pharmaceutically effective amount of a cyclic peptidomimetic disclosed herein, for example to treat cancer.

### BACKGROUND

### Technical Field

The present disclosure generally relates to compounds comprising a cyclic β-hairpin peptidomimetic suitable for use in treatment of various diseases, including cancer, and methods of making and using the same.

### Background

Multiple myeloma is a cancer of the plasma cell, which primarily develops in the elderly population. The progression of the tumor is well understood, and it can be diagnosed by the presence of multiple myeloma cells in the bone marrow and monitored by the amount of antibody secretion from the clonal population of plasma cells. A premalignant condition known as monoclonal gammopathy of undetermined significance (MGUS) develops at certain rates in the US population: 3% at age 50, 5% at age 70, and 7% by age 85; approximately 1% of MGUS patients progress to multiple myeloma on an annual basis (Kyle R. A., et. al, N. Engl. J. Med. 354, 1362-1369 (2006)). The molecular causes for progression from MGUS to multiple myeloma are unknown. Despite highly specific and easily detectable biomarkers, many challenges still exist for multiple myeloma treatment. While some treatments have had some success against multiple myeloma cells, many challenges exist as a barrier to finding effective treatment.

Accordingly, there is a need in the art for improved compounds having utility for treating the aforementioned diseases and methods for using and preparing the same. Embodiments of the present disclosure provide this and related improvements.

### BRIEF DESCRIPTION

Briefly, embodiments of the present disclosure relate to compounds comprising cyclic peptidomimetic compounds, as well as the preparation and use thereof. In particular, embodiments of compounds comprising cyclic β-hairpin peptidomimetic compounds disclosed herein have shown anti-cancer activity against cancer, such as multiple myeloma, EGFR-driven lung cancer, and prostate cancer. The applicant has discovered that some embodiments of these compounds with a linker, such as 3-aminopropyl and certain diverse side chains (e.g., substituted or unsubstituted amino acid derivatives), can significantly enhance the desired biological activity and bioavailability. In some embodiments, the applicant has unexpectedly discovered that attachment of a cysteine side chain significantly increases biological potency and activity, which is consistent with the additional positive charge from protonation of the cysteine N-terminus at physiological pH.

Accordingly, one embodiment of the present disclosure provides a compound having a structure of Formula (I):

X-L¹-N(R¹)R² (I)

or a pharmaceutically acceptable salt, tautomer, or stereoisomer thereof, wherein:
X is a cyclic β-hairpin peptidomimetic;
L¹ is an optional linker;
R¹ is H; and
R² is H or a moiety comprising one or more amino acid residues or substituted derivatives thereof.

Another embodiment provides a pharmaceutical composition comprising a compound according to the embodiments disclosed herein and a pharmaceutically acceptable carrier or excipient.

Yet another embodiment provides a method for treatment of cancer, the method comprising administering an effective amount of a pharmaceutical composition as disclosed herein to a subject in need thereof.

These and other aspects will be evident upon reference to the attached drawings and following detailed description.

### BRIEF DESCRIPTION OF THE DRAWINGS

In the FIGURES, identical reference numbers identify similar elements or acts. The sizes and relative positions of elements in the figures are not necessarily drawn to scale. For example, the shapes of various elements and angles are not drawn to scale and some of these elements are enlarged and positioned to improve FIGURE legibility. Further, the particular shapes of the elements as drawn, are not intended to convey any information regarding the actual shape of the particular elements, and have been solely selected for ease of recognition in the FIGURES.

FIG. 1 shows Compound 2 having increased potency relative to Compound A.

### DETAILED DESCRIPTION

The particulars described herein are by way of example and are only for purposes of illustrative discussion of embodiments of the present disclosure. The use of any and all examples, or exemplary language (e.g., “such as” or “for example”) provided herein is merely intended to better illuminate the disclosure and does not pose a limitation on the scope of the disclosure as claimed. No language in the specification should be construed as indicating any non-claimed element is essential to the practice of the disclosure. Further, all methods described herein can be performed in any suitable order unless otherwise indicated herein or otherwise clearly contradicted by context.

Definitions used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art.

The use of the alternative (e.g., “or”) should be understood to mean one, both, or any combination thereof of the alternatives. The various embodiments described above can be combined to provide further embodiments. Groupings of alternative elements or embodiments of the disclosure described herein should not be construed as limitations. Each member of a group may be referred to and claimed individually, or in any combination with other members of the group or other elements found herein.

Each embodiment disclosed herein can comprise, consist essentially of, or consist of a particular stated element, step, ingredient, or component. As used herein, the term “comprise” or “comprises” means “includes, but is not limited to,” and allows for the inclusion of unspecified elements, steps, ingredients, or components, even in major amounts. As used herein, the phrase “consisting of” excludes any element, step, ingredient, or component that is not specified. As used herein, the phrase “consisting essentially of” limits the scope of the embodiment to the specified elements, steps, ingredients, or components, and to those that do not materially affect the basic and novel characteristics of the claimed disclosure.

The terms “a,” “an,” “the,” and similar articles or terms used in the context of describing the disclosure (especially in the context of the following claims) are to be construed to cover both the singular and the plural (i.e., “one or more”), unless otherwise indicated herein or clearly contradicted by context. Ranges of values recited herein are intended to serve as a shorthand method of referring individually to each separate value falling within the range. In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as size or thickness, are to be understood to include any integer within the recited range, unless otherwise indicated. Unless otherwise indicated herein, each individual value is incorporated into the specification as if it were individually recited herein.

The term “about” has the meaning reasonably ascribed to it by a person of ordinary skill in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

“Amino” refers to the —NH₂ group.

“Hydroxy” or “hydroxyl” refers to the —OH group.

“Alkyl” refers to a straight or branched hydrocarbon chain group consisting solely of carbon and hydrogen atoms, containing no unsaturation, having from one to eighteen carbon atoms (C₁-C₁₈ alkyl), one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, alkyl groups are optionally substituted.

“Aminoalkyl” is an alkyl group comprising at least one amino substituent. The amino substituent may be on a primary, secondary or tertiary carbon. Unless stated otherwise specifically in the specification, aminoalkyl groups are optionally substituted.

“Hydroxyalkyl” or “hydroxylalkyl” refers to an alkyl group, as defined above, comprising at least one hydroxy substituent. The —OH substituent may be on a primary, secondary or tertiary carbon. Unless stated otherwise specifically in the specification, a hydroxyalkyl group is optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Heteroalkylene” refers to an alkylene group, as defined above, comprising at least one heteroatom (e.g., N, O, P or S) within the alkylene chain or at a terminus of the alkylene chain. In some embodiments, the heteroatom is within the alkylene chain (i.e., the heteroalkylene comprises at least one carbon-[heteroatom]ₓ-carbon bond, where x is 1, 2 or 3). In other embodiments, the heteroatom is at a terminus of the alkylene and thus serves to join the alkylene to the remainder of the molecule (e.g., M1-Hₐ-A-M2, where M1 and M2 are portions of the molecule, Hₐ is a heteroatom and A is an alkylene). Unless stated otherwise specifically in the specification, a heteroalkylene group is optionally substituted.

“Protecting group” refers to a chemical used to protect a particular functional group during desired synthetic steps. Functional groups may include, e.g., hydroxy, amino, and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino (i.e., an “amine protecting group”), amidino and guanidino include an allyloxy carbonyl protecting group, a benzyloxycarbonyl protecting group, a butyloxycarbonyl protecting group (e.g., t-butoxycarbonyl or Boc), or a fluorenylmethyloxycarbonyl protecting group (or Fmoc) and the like. For example, A Boc protected amine or an Fmoc protected amine would have the following structures, respectively:

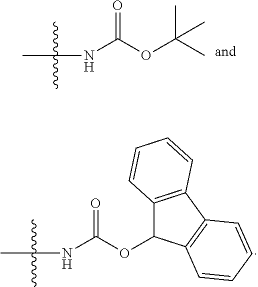

Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3^{rd} Ed., Wiley. As one of skill in the art would appreciate, the protecting group may also be a polymer resin such as a Wang resin, Rink resin or a 2-chlorotrityl-chloride resin. In some embodiments, protecting groups include, e.g., a triphenylmethyl protecting group, a dimethoxytriphenylmethyl protecting group, allyloxycarbonyl protecting group, a benzyloxycarbonyl protecting group, a butyloxycarbonyl protecting group, or a fluorenylmethyloxycarbonyl protecting group.

A “linker” refers to a contiguous chain of at least one atom, such as carbon, oxygen, nitrogen, sulfur, phosphorous and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions.

“Sarcosine” refers to N-methylglycine, which is an amino acid having the following structure:

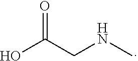

When sarcosine is included in an amino acid sequence, it may have one of the following structures:

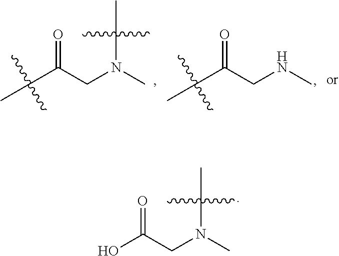

“Norleucine” (abbreviated Nle or N*) or 2-aminohexanoic acid is an amino acid compound that is a isomer of leucine and has the following structure:

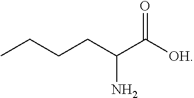

When norleucine is included in an amino acid sequence, it may have one of the following structures:

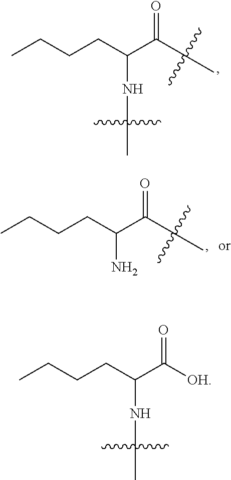

“Saccharide” refers to a group of compounds including sugars, starch, and cellulose, monosaccharides, disaccharides, oligosaccharides, and polysaccharides and can exist in ring or short chain conformation and generally have the molecular formula Cₘ(H₂O)ₙ wherein m may be different than n (with some exceptions such as deoxyribose). Saccharides include, e.g., moieties having one of the following structures (and stereoisomers thereof):

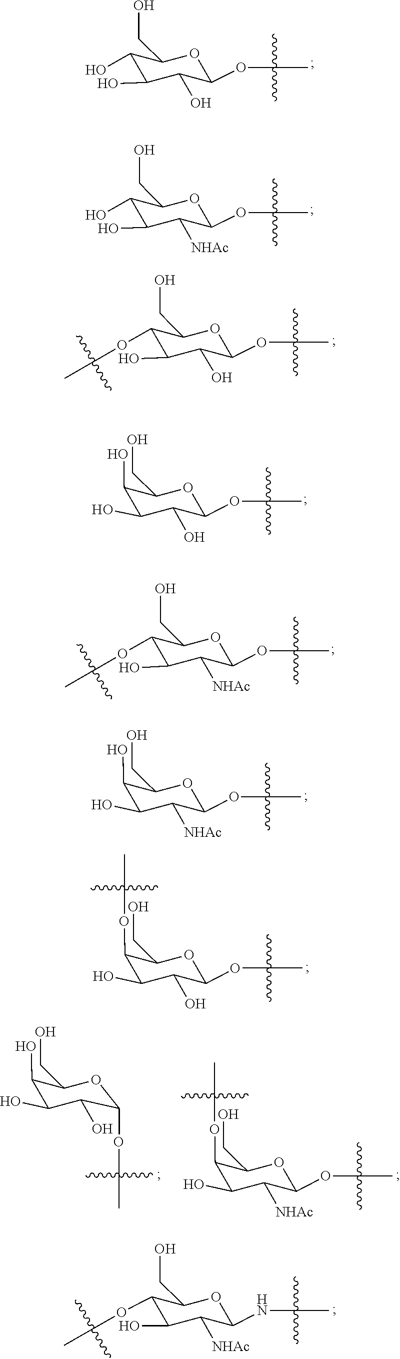

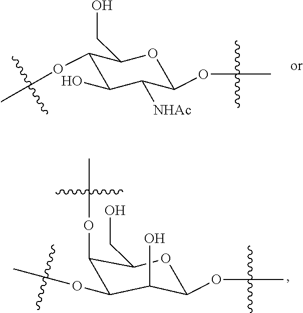 [SMILES: *OC1OC(CO)C(O*)C(O)C1NC(C)=O ; *OC1OC(CO)C(O*)C(O*)C1O]

“Glycosidic bond” or “glycosidic linkage” refers to a type of covalent bond that joins a saccharide molecule or moiety to another group that may or may not be a saccharide.

The subjects that can be treated according to the methods of the current disclosure can be a human or non-human animal. For example, the subject can be a human, non-human primate, pig, dog, rodent, feline, bovine, or other mammal. As used herein, the terms “subject” and “patient” are used interchangeably. Likewise, the compounds and compositions can be administered to human cells or non-human animal cells in vitro or in vivo. In some embodiments, the cells are mammalian cells.

The pharmaceutically effective amount of the compound depends on the type of disease to be treated as well as the tolerance of the subject for the treatment.

The disease treatment according to the current disclosure can also be administered alone or in combination with one or more other treatments. For example, cancer in a subject can be treated by administering a compound or composition of the current disclosure in combination (simultaneously or consecutively) with chemotherapy and/or radiotherapy. For some diseases, treatment of the subject may include surgery. The compound or composition may be administered before or after surgery.

As used herein, the terms “treat” or “treatment” refer to both therapeutic treatment and prophylactic or preventative measures, wherein the object is to prevent or slow down (lessen) an undesired physiological change or disorder, such as the development or spread of an oncological disorder (e.g., cancer). In some embodiments, the subject has a cancer at the time of administration. In other embodiments, the subject does not have a cancer at the time of administration, in which case the compound or composition may be administered to prevent or delay onset of the cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. In some embodiments, the treatment methods include identifying the subject as having cancer or another disease or disorder to be treated.

The amount of the compound (e.g., a compound of Formula (I)) or composition administered to the subject or cell may be an effective amount, e.g., a therapeutically effective amount. As used herein, the term “(therapeutically) effective amount” refers to an amount of an agent (e.g., a compound or composition of the disclosure) effective to treat a disease or disorder in a subject. In the case of cancer, the therapeutically effective amount of the agent may directly or indirectly (e.g., through an immune response) reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

While the compounds (e.g., compounds of Formula (I)) can be administered as isolated compounds, these compounds can also be administered as part of a pharmaceutical composition. The subject disclosure provides compositions comprising one or more compounds of the current disclosure in association with at least one pharmaceutically acceptable carrier. Compounds and compositions containing them can be administered to a subject locally at or adjacent to a site of intended action (e.g., a tumor or lesion), or systemically (e.g., intravascularly). The compound and pharmaceutical composition can be adapted for various routes of administration, such as enteral, parenteral, intravenous, intramuscular, topical, subcutaneous, and so forth. Administration can be continuous or at distinct intervals, as can be determined by a person of ordinary skill in the art. Optionally, the suitable bioactive agents that are optionally administered with the compounds separately or within the same formulation can be formulated as pharmaceutically acceptable salts or solvates.

Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example, reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids can also be made.

A “linker” refers to a contiguous chain of at least one atom, such as carbon, oxygen, nitrogen, sulfur, phosphorous and combinations thereof, which connects a portion of a molecule (e.g., a cyclic β-hairpin peptidomimetic) to a different molecule or molecules, moiety or moieties, or a solid support (e.g., amino acid residues or derivatives thereof). Linkers may connect the different agents, moieties or molecules via a covalent bond or other means, such as ionic or hydrogen bond interactions. Linkers can be branched to connect one molecule to a plurality of different molecules.

An “amino acid” or “amino acid residue” refers to an α-amino acid residue (—CO—CHR—NH—), where R is a side chain. Amino acids are denoted with a 3-letter or 1-letter code according to the table below:

| Amino Acid | 3-Letter Code | 1-Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “naturally occurring amino acid” refers to an amino acid present in proteins found in nature. Examples of naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL) and anti-signaling agents (e.g., the PI3K inhibitor LY).

### A. Compounds

In one embodiment the disclosure relates to a compound comprising a cyclic β-hairpin peptidomimetic, an optional linker and optionally one or more amino acid residues or derivatives thereof. Accordingly, some embodiments provide a compound having a structure of Formula (I):

X-L¹-N(R¹)R², (I)

or a pharmaceutically acceptable salt, tautomer, or stereoisomer thereof, wherein:
X is a cyclic β-hairpin peptidomimetic;
L¹ is an optional linker;
R¹ is H; and
R² is H or a moiety comprising one or more amino acid residues or substituted derivatives thereof.

In some embodiments, L¹ is absent. In certain embodiments, L¹ is a heteroalkylene linker. For example, in some embodiments, the heteroalkylene linker comprises from 2 to 6 carbons and a heteroatom selected from O, S, N and P. In some embodiments, L¹ is optionally substituted.

In some more specific embodiments, L¹ is —S(O)ₜ—(CH₂)ₙ—, wherein:
t is 0, 1 or 2; and
n is 1, 2, 3, 4, 5 or 6.

In some embodiments, t is 2. In some embodiments, t is 1. In some embodiments, t is 0. In certain embodiments, n is 3, 4, 5 or 6. In more specific embodiments, n is 3. In certain specific embodiments, L¹ has the following structure:

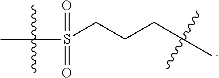 [SMILES: *CCCS(*)(=O)=O]

In some embodiments of the foregoing, R² is H. In certain related embodiments, R² comprises one or more amino acid residues or substituted derivatives thereof. In more specific embodiments, R² comprises one or more cysteine residues or derivatives thereof. In some embodiments, R² comprises one or two cysteine residues or derivatives thereof.

In other embodiments, R² has one of the following structures:

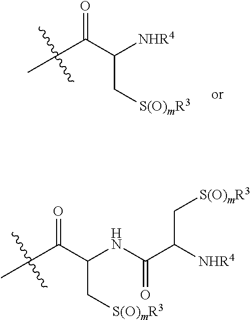

wherein:
R³ is, at each occurrence, independently H, a protecting group, alkyl, aminoalkyl, a protected aminoalkyl, hydroxyalkyl, a protected hydroxyalkyl, or -L²-Y;
R⁴ is H or an amine protecting group;
m is, at each occurrence, independently, 0, 1 or 2;
L² is a linker; and
Y at each occurrence independently comprises one or more saccharide moieties.

In some embodiments, R² has one of the following structures:

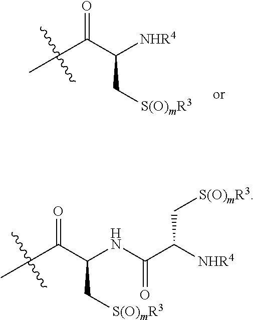

In some specific embodiments, R² has one of the following structures:

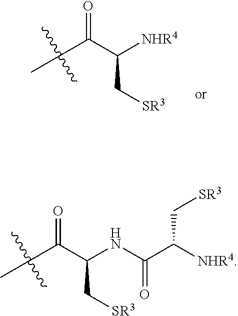

In certain specific embodiments, R² has one of the following structures:

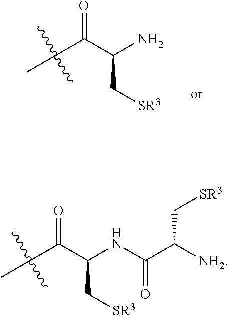

In certain specific embodiments, R² has one of the following structures:

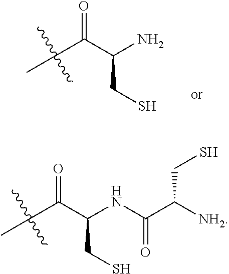 [SMILES: *C(=O)[C@@H](N)CS ; *C(=O)[C@H](CS)NC(=O)[C@@H](N)CS]

In some embodiments, at least one occurrence of R³ is H. In some related embodiments, R³ is H at each occurrence.

In other embodiments, at least one occurrence of R³ is a protecting group. In some embodiments, R³ is a protecting group at each occurrence. For example, in some embodiments, the protecting group is a triphenylmethyl protecting group or a dimethoxytriphenylmethyl protecting group.

In other embodiments, at least one occurrence of R³ is a protected aminoalkyl or a protected hydroxyalkyl. In more specific embodiments, R³ is a protected aminoalkyl or a protected hydroxyalkyl at each occurrence. For example, in some embodiments, the protected aminoalkyl or protected hydroxyalkyl comprises an allyloxy carbonyl protecting group, a benzyloxy carbonyl protecting group, a butyloxy carbonyl protecting group, or a fluorenylmethyloxy carbonyl protecting group.

In some of the foregoing embodiments, at least one occurrence of R³ is C₁-C₁₈ alkyl, C₁-C₁₈ aminoalkyl, a protected C₁-C₁₈ aminoalkyl, a protected C₁-C₁₈ hydroxyalkyl or C₁-C₁₈ hydroxyalkyl. In more specific embodiments, R³ is C₁-C₁₈ alkyl, C₁-C₁₈ aminoalkyl, a protected C₁-C₁₈ aminoalkyl, a protected C₁-C₁₈ hydroxyalkyl or C₁-C₁₈ hydroxyalkyl at each occurrence.

In some of the foregoing embodiments, at least one occurrence of R³ is C₁-C₁₂ alkyl, C₁-C₁₂ aminoalkyl, a protected C₁-C₁₂ aminoalkyl, a protected C₁-C₁₂ hydroxyalkyl or C₁-C₁₂ hydroxyalkyl. In more specific embodiments, R³ is C₁-C₁₂ alkyl, C₁-C₁₂ aminoalkyl, a protected C₁-C₁₂ aminoalkyl, a protected C₁-C₁₂ hydroxyalkyl or C₁-C₁₂ hydroxyalkyl at each occurrence.

In some of the foregoing embodiments, at least one occurrence of R³ is C₁-C₈ alkyl, C₁-C₈ aminoalkyl, a protected C₁-C₈ aminoalkyl, a protected C₁-C₈ hydroxyalkyl or C₁-C₈ hydroxyalkyl. In more specific embodiments, R³ is C₁-C₈ alkyl, C₁-C₈ aminoalkyl, a protected C₁-C₈ aminoalkyl, a protected C₁-C₈ hydroxyalkyl or C₁-C₈ hydroxyalkyl at each occurrence.

In some embodiments, at least one occurrence of R³ is -L²-Y. In more specific embodiments, R³ is -L²-Y at each occurrence. In certain specific embodiments, L² has the following structure:

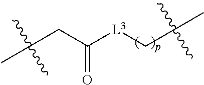

wherein:
L³ is a direct bond or —NH—; and
p is 0, 1 or 2.

For example, in certain embodiments, L² has one of the following structures:

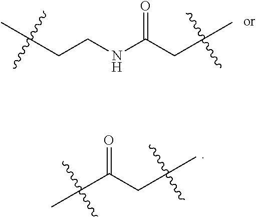 [SMILES: *CCNC(=O)C* ; *CC(*)=O]

In certain related embodiments, Y comprises one of the following saccharide moieties:

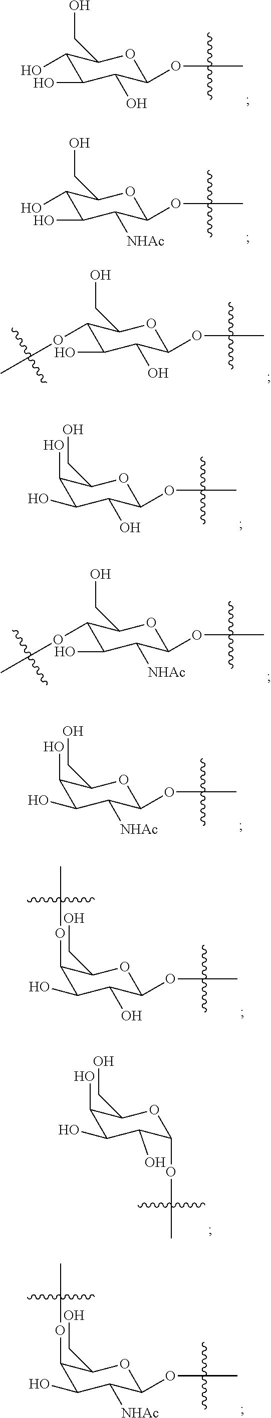

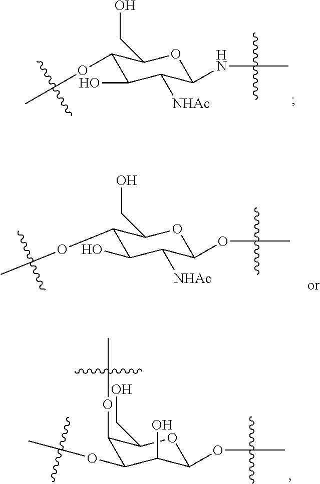 [SMILES: *NC1OC(CO)C(O*)C(O)C1NC(C)=O ; *OC1OC(CO)C(O*)C(O)C1NC(C)=O ; *OC1OC(CO)C(O*)C(O*)C1O]

wherein:

[symbol] at each occurrence independently indicates a linkage to L², a bond to a H, or a glycosidic link to a saccharide moiety optionally substituted with one or more additional saccharide moieties, provided that at least one [symbol] is a linkage to L².

In more specific embodiments, Y comprises one of the following saccharide moieties:

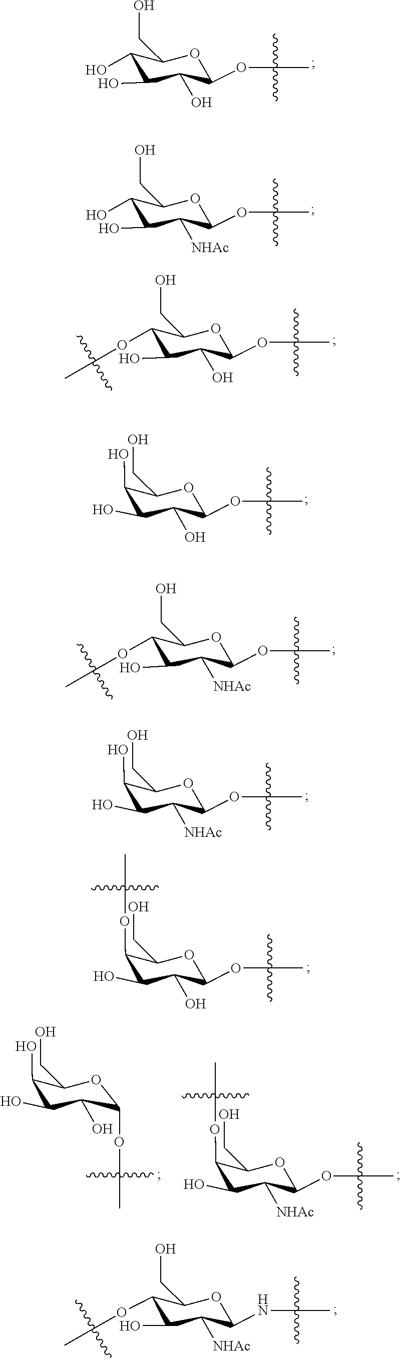

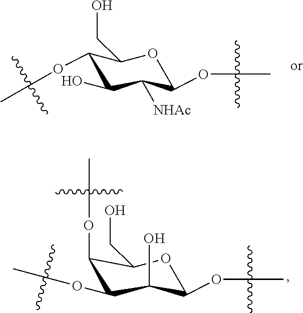

wherein:

[symbol] at each occurrence independently indicates a linkage to L², a bond to a H, or a glycosidic link to a saccharide moiety optionally substituted with one or more additional saccharide moieties, provided that at least one [symbol] is a linkage to L². For example, in some embodiments, Y comprises 1, 2, 3, 4, 5, 6, 7, 8, or 9 saccharide moieties.

In more specific embodiments of the foregoing, -L²-Y has one of the following structures:

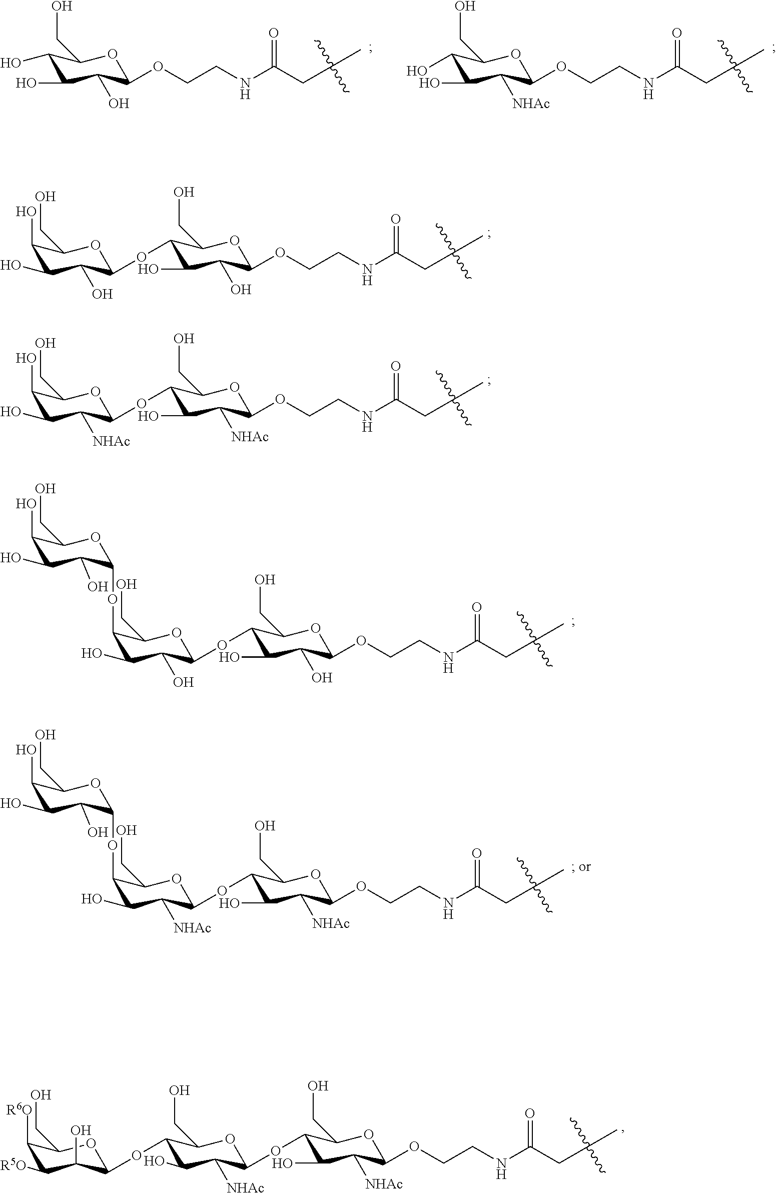

wherein:

R⁵ and R⁶ are, at each occurrence, independently H, or a glycosidic link to a saccharide moiety optionally substituted with one or more additional saccharide moieties.

In some embodiments, R⁴ is an amine protecting group. For example, in some embodiments, R⁴ is a butyloxycarbonyl protecting group, or a fluorenylmethyloxycarbonyl protecting group. In other embodiments, R⁴ is H.

In some embodiments, m is 0. In other embodiments, m is 1. In still other embodiments, m is 2.

In some embodiments, the cyclic β-hairpin peptidomimetic comprises a recognition sequence and a non-recognition sequence and the recognition sequence is linked to the non-recognition sequence by a first linker and a second linker. In some more specific embodiment, the non-recognition sequence is 5 amino acids selected from KLKLK (SEQ ID NO:27), KLQLK (SEQ ID NO:28), QLKLK (SEQ ID NO:29), KLKLQ (SEQ ID NO:281), KQKLK (SEQ ID NO:30), KLKQK (SEQ ID NO:282), KXKXK (SEQ ID NO:31), or ELKLK (SEQ ID NO:32), wherein X is sarcosine.

In certain embodiments, the recognition sequence is five amino acids selected from MVVSW (SEQ ID NO:33), MVVSA (SEQ ID NO:34), MVVAW (SEQ ID NO: 35), MVASW (SEQ ID NO:36), MAVSW (SEQ ID NO:37), AVVSW (SEQ ID NO: 38), N*VVSW (SEQ ID NO:39), N*VVYW (SEQ ID NO:40), N*VVAW (SEQ ID NO: 41), AVVAW (SEQ ID NO:42), N*AVAW (SEQ ID NO:43), N*VAAW (SEQ ID NO: 44), N*VLAW (SEQ ID NO:45), N*VIAW (SEQ ID NO:46), N*VFAW (SEQ ID NO: 47), or WSVVW (SEQ ID NO:48), WAVAW (SEQ ID NO:50), WAVAA (SEQ ID NO: 51), WAVAM (SEQ ID NO:52), WAVAN* (SEQ ID NO:53), WAVVN* (SEQ ID NO: 54), WAVSN* (SEQ ID NO:55), WAAAW (SEQ ID NO:56), WAAAA (SEQ ID NO: 57), WAAAM (SEQ ID NO:58), WAAAN* (SEQ ID NO:59), WAAVW (SEQ ID NO: 60), WAAVA (SEQ ID NO:61), WAAVM (SEQ ID NO:62), WAAVN* (SEQ ID NO: 63), WAASN* (SEQ ID NO:64), WVVAW (SEQ ID NO:65), WVVAA (SEQ ID NO: 66), WVVAM (SEQ ID NO:67), WVVAN* (SEQ ID NO:68), WVVVW (SEQ ID NO: 69), WVVVA (SEQ ID NO:70), WVVVM (SEQ ID NO:71), WVVVN* (SEQ ID NO: 72), WVVSN* (SEQ ID NO:73), WVAAN* (SEQ ID NO:74), WVAVW (SEQ ID NO: 75), WVAVA (SEQ ID NO:76), WVAVM (SEQ ID NO:77), WVAVN* (SEQ ID NO: 78), WVASN* (SEQ ID NO:79), WSVAW (SEQ ID NO:80), WSVAA (SEQ ID NO: 81), WSVAM (SEQ ID NO:82), WSVAN* (SEQ ID NO:83), WSVVW (SEQ ID NO: 48), WSVVA (SEQ ID NO:84), WSVVM (SEQ ID NO:85), WSVVN* (SEQ ID NO: 86), WSVSW (SEQ ID NO:87), WSVSA (SEQ ID NO:88), WSVSM (SEQ ID NO: 89), WSVSN* (SEQ ID NO:90), WSAAW (SEQ ID NO:91), WSAAA (SEQ ID NO: 92), WSAAM (SEQ ID NO:93), WSAAN* (SEQ ID NO:94), WSAVW (SEQ ID NO: 95), WSAVA (SEQ ID NO:96), WSAVM (SEQ ID NO:97), WSAVN* (SEQ ID NO: 98), WSASW (SEQ ID NO:99), WSASA (SEQ ID NO:100), WSASM (SEQ ID NO: 101), WSASN* (SEQ ID NO:102), WYVAW (SEQ ID NO:103), WYVAA (SEQ ID NO: 104), WYVAM (SEQ ID NO:105), WYVAN* (SEQ ID NO:106), WYVVW (SEQ ID NO:107), WYVVA (SEQ ID NO:108), WYVVM (SEQ ID NO:109), WYVVN* (SEQ ID NO: 110), WYVSW (SEQ ID NO:111), WYVSA (SEQ ID NO: 112), WYVSM (SEQ ID NO:113), WYVSN* (SEQ ID NO:114), WYAAW (SEQ ID NO: 115), WYAAA (SEQ ID NO:116), WYAAM (SEQ ID NO:117), WYAAN* (SEQ ID NO:118), WYAAW (SEQ ID NO:119), WYAVA (SEQ ID NO:120), WYAVM (SEQ ID NO:121), WYAVN* (SEQ ID NO:122), WYASW (SEQ ID NO:123), WYASA (SEQ ID NO:124), WYASM (SEQ ID NO:125), WYASN* (SEQ ID NO:126), AAVAA (SEQ ID NO:127), AAVAM (SEQ ID NO:128), AAVAN* (SEQ ID NO:129), AAVVN* (SEQ ID NO:130), AAVAN* (SEQ ID NO:131), AAAAA (SEQ ID NO:132), AAAAM (SEQ ID NO:133), AAAAN* (SEQ ID NO:134), AAAVW (SEQ ID NO:135), AAAVA (SEQ ID NO:136), AAAVM (SEQ ID NO:137), AAAVN* (SEQ ID NO:138), AAASM (SEQ ID NO:139), AAASN* (SEQ ID NO:140), AVVAW (SEQ ID NO:141), AVVAA (SEQ ID NO: 142), AVVAM (SEQ ID NO:143), AVVAN* (SEQ ID NO:144), AVVVA (SEQ ID NO:145), AVVVM (SEQ ID NO:146), AVVVN* (SEQ ID NO:147), AVVSN* (SEQ ID NO:148), AVAAW (SEQ ID NO:42), AVAAM (SEQ ID NO:149), AVAAN* (SEQ ID NO:150), AVAVA (SEQ ID NO:151), AVAVM (SEQ ID NO:152), AVAVN* (SEQ ID NO:153), AVASN* (SEQ ID NO:154), ASVAW (SEQ ID NO:155), ASVAA (SEQ ID NO:156), ASVAM (SEQ ID NO:157), ASVAN* (SEQ ID NO:158), ASVVW (SEQ ID NO:159), ASVVA (SEQ ID NO:160), ASVVM (SEQ ID NO:161), ASVVN* (SEQ ID NO:162), ASVSA (SEQ ID NO:163), ASVSM (SEQ ID NO:164), ASVSN* (SEQ ID NO:165), ASAAW (SEQ ID NO:166), ASAAA (SEQ ID NO:167), ASAAM (SEQ ID NO:168), ASAAN* (SEQ ID NO:169), ASAVW (SEQ ID NO:170), ASAVA (SEQ ID NO:171), ASAVM (SEQ ID NO:172), ASAVN* (SEQ ID NO:173), ASASA (SEQ ID NO:174), ASASM (SEQ ID NO:175), ASASN* (SEQ ID NO:176), AYVAW (SEQ ID NO:177), AYVAA (SEQ ID NO:178), AYVAM (SEQ ID NO:179), AYVAN* (SEQ ID NO:180), AYVVW (SEQ ID NO:181), AYVVA (SEQ ID NO:182), AYVVM (SEQ ID NO:183), AYVVN* (SEQ ID NO:184), AYVSW (SEQ ID NO:185), AYVSA (SEQ ID NO:186), AYVSM (SEQ ID NO:187), AYVSN* (SEQ ID NO:188), AYAAW (SEQ ID NO:189), AYAAA (SEQ ID NO:190), AYAAM (SEQ ID NO:191), AYAAN* (SEQ ID NO:192), AYAVW (SEQ ID NO:193), AYAVA (SEQ ID NO:194), AYAVM (SEQ ID NO:195), AYAVN* (SEQ ID NO:196), AYASW (SEQ ID NO:197), AYASA (SEQ ID NO:198), AYASM (SEQ ID NO:199), AYASN* (SEQ ID NO:200), MAVAA (SEQ ID NO:201), MAVAM (SEQ ID NO:202), MAVAN* (SEQ ID NO:203), MAVVN* (SEQ ID NO:204), MAVSN* (SEQ ID NO:205), MAAAA (SEQ ID NO: 206), MAAAM (SEQ ID NO:207), MAAAN* (SEQ ID NO:208), MAAVW (SEQ ID NO: 209), MAAVA (SEQ ID NO:210), MAAVM (SEQ ID NO:211), MAAVN* (SEQ ID NO: 212), MAASN* (SEQ ID NO:213), MVVAW (SEQ ID NO:214), MVVAA (SEQ ID NO: 215), MVVAM (SEQ ID NO:216), MVVAN* (SEQ ID NO:217), MVVVM (SEQ ID NO:218), MVVVN* (SEQ ID NO:219), MVVSN* (SEQ ID NO:220), MVAAM (SEQ ID NO:221), MVAAN* (SEQ ID NO:222), MVAVM (SEQ ID NO: 223), MVAVN* (SEQ ID NO:224), MVASN* (SEQ ID NO:225), MSVAW (SEQ ID NO: 226), MSVAA (SEQ ID NO:227), MSVAM (SEQ ID NO:228), MSVAN* (SEQ ID NO: 229), MSVVW (SEQ ID NO:230), MSVVA (SEQ ID NO:231), MSVVM (SEQ ID NO: 232), MSVVN* (SEQ ID NO:233), MSVSM (SEQ ID NO:234), MSVSN* (SEQ ID NO: 235), MSAAW (SEQ ID NO:236), MSAAA (SEQ ID NO:237), MSAAM (SEQ ID NO: 238), MSAAN* (SEQ ID NO:239), MSAVW (SEQ ID NO:240), MSAVA (SEQ ID NO: 241), MSAVM (SEQ ID NO:242), MSAVN* (SEQ ID NO:243), MSASM (SEQ ID NO: 244), MSASN* (SEQ ID NO:245), MYVAW (SEQ ID NO:246), MYVAA (SEQ ID NO: 247), MYVAM (SEQ ID NO:248), MYVAN* (SEQ ID NO:249), MYVVW (SEQ ID NO:250), MYVVA (SEQ ID NO:251), MYVVM (SEQ ID NO:252), MYVVN* (SEQ ID NO:253), MYVSW (SEQ ID NO:254), MYVSA (SEQ ID NO:255), MYVSM (SEQ ID NO:256), MYVSN* (SEQ ID NO:257), MYAAW (SEQ ID NO:258), MYAAA (SEQ ID NO:259), MYAAM (SEQ ID NO:260), MYAAN* (SEQ ID NO:261), MYAVW (SEQ ID NO:262), MYAVA (SEQ ID NO:263), MYAVM (SEQ ID NO:264), MYAVN* (SEQ ID NO:265), MYASW (SEQ ID NO:266), MYASA (SEQ ID NO:267), MYASM (SEQ ID NO:268), or MYASN* (SEQ ID NO:269), wherein N* is norleucine.

In some more specific embodiments, the recognition sequence is five amino acids selected from MVVSW (SEQ ID NO:33), MVVSA (SEQ ID NO:34), MVVAW (SEQ ID NO:35), MVASW (SEQ ID NO:36), MAVSW (SEQ ID NO:37), AVVSW (SEQ ID NO:38), N*VVSW (SEQ ID NO:39), N*VVYW (SEQ ID NO:40), N*VVAW (SEQ ID NO:41), AVVAW (SEQ ID NO:42), N*AVAW (SEQ ID NO:43), N*VAAW (SEQ ID NO:44), N*VLAW (SEQ ID NO:45), N*VIAW (SEQ ID NO:46), N*VFAW (SEQ ID NO:47), or WSVVW (SEQ ID NO:48), wherein N* is norleucine.

In some embodiments, the non-recognition sequence is KLKLK (SEQ ID NO: 27). In some specific embodiments, the recognition sequence is N*VVAW (SEQ ID NO: 41), wherein N* is norleucine. In certain embodiments, the non-recognition sequence is KLKLK (SEQ ID NO:27) and the recognition sequence is N*VVAW (SEQ ID NO: 41), wherein N* is norleucine. The sequence identification numbers (i.e., SEQ ID Nos) are as set forth in Table 1 below.

TABLE 1
Sequence identification numbers

| Sequence | Amino Acid |
|---|---|
| Identification | Sequence |
| Number | (with 1-letter |
| (SEQ ID NO) | codes)^{†,} ^{‡} |
| SEQ ID NO: 27 | KLKLK |
| SEQ ID NO: 28 | KLQLK |
| SEQ ID NO: 29 | QLKLK |
| SEQ ID NO: 30 | KQKLK |
| SEQ ID NO: 31 | KXKXK |
| SEQ ID NO: 32 | ELKLK |
| SEQ ID NO: 33 | MVVSW |
| SEQ ID NO: 34 | MVVSA |
| SEQ ID NO: 35 | MVVAW |
| SEQ ID NO: 36 | MVASW |
| SEQ ID NO: 37 | MAVSW |
| SEQ ID NO: 38 | AVVSW |
| SEQ ID NO: 39 | N*VVSW |
| SEQ ID NO: 40 | N*VVYW |
| SEQ ID NO: 41 | N*VVAW |
| SEQ ID NO: 42 | AVVAW |
| SEQ ID NO: 43 | N*AVAW |
| SEQ ID NO: 44 | N*VAAW |
| SEQ ID NO: 45 | N*VLAW |
| SEQ ID NO: 46 | N*VIAW |
| SEQ ID NO: 47 | N*VFAW |
| SEQ ID NO: 48 | WSVVW |
| SEQ ID NO: 50 | WAVAW |
| SEQ ID NO: 51 | WAVAA |
| SEQ ID NO: 52 | WAVAM |
| SEQ ID NO: 53 | WAVAN* |
| SEQ ID NO: 54 | WAVVN* |
| SEQ ID NO: 55 | WAVSN* |
| SEQ ID NO: 56 | WAAAW |
| SEQ ID NO: 57 | WAAAA |
| SEQ ID NO: 58 | WAAAM |
| SEQ ID NO: 59 | WAAAN* |
| SEQ ID NO: 60 | WAAVW |
| SEQ ID NO: 61 | WAAVA |
| SEQ ID NO: 62 | WAAVM |
| SEQ ID NO: 63 | WAAVN* |
| SEQ ID NO: 64 | WAASN* |
| SEQ ID NO: 65 | WVVAW |
| SEQ ID NO: 66 | WVVAA |
| SEQ ID NO: 67 | WVVAM |
| SEQ ID NO: 68 | WVVAN* |
| SEQ ID NO: 69 | WVVVW |
| SEQ ID NO: 70 | WVVVA |
| SEQ ID NO: 71 | WVVVM |
| SEQ ID NO: 72 | WVVVN* |
| SEQ ID NO: 73 | WVVSN* |
| SEQ ID NO: 74 | WVAAN* |
| SEQ ID NO: 75 | WVAVW |
| SEQ ID NO: 76 | WVAVA |
| SEQ ID NO: 77 | WVAVM |
| SEQ ID NO: 78 | WVAVN* |
| SEQ ID NO: 79 | WVASN* |
| SEQ ID NO: 80 | WSVAW |
| SEQ ID NO: 81 | WSVAA |
| SEQ ID NO: 82 | WSVAM |
| SEQ ID NO: 83 | WSVAN* |
| SEQ ID NO: 84 | WSVVA |
| SEQ ID NO: 85 | WSVVM |
| SEQ ID NO: 86 | WSVVN* |
| SEQ ID NO: 87 | WSVSW |
| SEQ ID NO: 88 | WSVSA |
| SEQ ID NO: 89 | WSVSM |
| SEQ ID NO: 90 | WSVSN* |
| SEQ ID NO: 91 | WSAAW |
| SEQ ID NO: 92 | WSAAA |
| SEQ ID NO: 93 | WSAAM |
| SEQ ID NO: 94 | WSAAN* |
| SEQ ID NO: 95 | WSAVW |
| SEQ ID NO: 96 | WSAVA |
| SEQ ID NO: 97 | WSAVM |
| SEQ ID NO: 98 | WSAVN* |
| SEQ ID NO: 99 | WSASW |
| SEQ ID NO: 100 | WSASA |
| SEQ ID NO: 101 | WSASM |
| SEQ ID NO: 102 | WSASN* |
| SEQ ID NO: 103 | WYVAW |
| SEQ ID NO: 104 | WYVAA |
| SEQ ID NO: 105 | WYVAM |
| SEQ ID NO: 106 | WYVAN* |
| SEQ ID NO: 107 | WYVVW |
| SEQ ID NO: 108 | WYVVA |
| SEQ ID NO: 109 | WYVVM |
| SEQ ID NO: 110 | WYVVN* |
| SEQ ID NO: 111 | WYVSW |
| SEQ ID NO: 112 | WYVSA |
| SEQ ID NO: 113 | WYVSM |
| SEQ ID NO: 114 | WYVSN* |
| SEQ ID NO: 115 | WYAAW |
| SEQ ID NO: 116 | WYAAA |
| SEQ ID NO: 117 | WYAAM |
| SEQ ID NO: 118 | WYAAN* |
| SEQ ID NO: 119 | WYAAW |
| SEQ ID NO: 120 | WYAVA |
| SEQ ID NO: 121 | WYAVM |
| SEQ ID NO: 122 | WYAVN* |
| SEQ ID NO: 123 | WYASW |
| SEQ ID NO: 124 | WYASA |
| SEQ ID NO: 125 | WYASM |
| SEQ ID NO: 126 | WYASN* |
| SEQ ID NO: 127 | AAVAA |
| SEQ ID NO: 128 | AAVAM |
| SEQ ID NO: 129 | AAVAN* |
| SEQ ID NO: 130 | AAVVN* |
| SEQ ID NO: 131 | AAVAN* |
| SEQ ID NO: 132 | AAAAA |
| SEQ ID NO: 133 | AAAAM |
| SEQ ID NO: 134 | AAAAN* |
| SEQ ID NO: 135 | AAAVW |
| SEQ ID NO: 136 | AAAVA |
| SEQ ID NO: 137 | AAAVM |
| SEQ ID NO: 138 | AAAVN* |
| SEQ ID NO: 139 | AAASM |
| SEQ ID NO: 140 | AAASN* |
| SEQ ID NO: 141 | AVVAW |
| SEQ ID NO: 142 | AVVAA |
| SEQ ID NO: 143 | AVVAM |
| SEQ ID NO: 144 | AVVAN* |
| SEQ ID NO: 145 | AVVVA |
| SEQ ID NO: 146 | AVVVM |
| SEQ ID NO: 147 | AVVVN* |
| SEQ ID NO: 148 | AVVSN* |
| SEQ ID NO: 149 | AVAAM |
| SEQ ID NO: 150 | AVAAN* |
| SEQ ID NO: 151 | AVAVA |
| SEQ ID NO: 152 | AVAVM |
| SEQ ID NO: 153 | AVAVN* |
| SEQ ID NO: 154 | AVASN* |
| SEQ ID NO: 155 | ASVAW |
| SEQ ID NO: 156 | ASVAA |
| SEQ ID NO: 157 | ASVAM |
| SEQ ID NO: 158 | ASVAN* |
| SEQ ID NO: 159 | ASVVW |
| SEQ ID NO: 160 | ASVVA |
| SEQ ID NO: 161 | ASVVM |
| SEQ ID NO: 162 | ASVVN* |
| SEQ ID NO: 163 | ASVSA |
| SEQ ID NO: 164 | ASVSM |
| SEQ ID NO: 165 | ASVSN* |
| SEQ ID NO: 166 | ASAAW |
| SEQ ID NO: 167 | ASAAA |
| SEQ ID NO: 168 | ASAAM |
| SEQ ID NO: 169 | ASAAN* |
| SEQ ID NO: 170 | ASAVW |
| SEQ ID NO: 171 | ASAVA |
| SEQ ID NO: 172 | ASAVM |
| SEQ ID NO: 173 | ASAVN* |
| SEQ ID NO: 174 | ASASA |
| SEQ ID NO: 175 | ASASM |
| SEQ ID NO: 176 | ASASN* |
| SEQ ID NO: 177 | AYVAW |
| SEQ ID NO: 178 | AYVAA |
| SEQ ID NO: 179 | AYVAM |
| SEQ ID NO: 180 | AYVAN* |
| SEQ ID NO: 181 | AYVVW |
| SEQ ID NO: 182 | AYVVA |
| SEQ ID NO: 183 | AYVVM |
| SEQ ID NO: 184 | AYVVN* |
| SEQ ID NO: 185 | AYVSW |
| SEQ ID NO: 186 | AYVSA |
| SEQ ID NO: 187 | AYVSM |
| SEQ ID NO: 188 | AYVSN* |
| SEQ ID NO: 189 | AYAAW |
| SEQ ID NO: 190 | AYAAA |
| SEQ ID NO: 191 | AYAAM |
| SEQ ID NO: 192 | AYAAN* |
| SEQ ID NO: 193 | AYAVW |
| SEQ ID NO: 194 | AYAVA |
| SEQ ID NO: 195 | AYAVM |
| SEQ ID NO: 196 | AYAVN* |
| SEQ ID NO: 197 | AYASW |
| SEQ ID NO: 198 | AYASA |
| SEQ ID NO: 199 | AYASM |
| SEQ ID NO: 200 | AYASN* |
| SEQ ID NO: 201 | MAVAA |
| SEQ ID NO: 202 | MAVAM |
| SEQ ID NO: 203 | MAVAN* |
| SEQ ID NO: 204 | MAVVN* |
| SEQ ID NO: 205 | MAVSN* |
| SEQ ID NO: 206 | MAAAA |
| SEQ ID NO: 207 | MAAAM |
| SEQ ID NO: 208 | MAAAN* |
| SEQ ID NO: 209 | MAAVW |
| SEQ ID NO: 210 | MAAVA |
| SEQ ID NO: 211 | MAAVM |
| SEQ ID NO: 212 | MAAVN* |
| SEQ ID NO: 213 | MAASN* |
| SEQ ID NO: 214 | MVVAW |
| SEQ ID NO: 215 | MVVAA |
| SEQ ID NO: 216 | MVVAM |
| SEQ ID NO: 217 | MVVAN* |
| SEQ ID NO: 218 | MVVVM |
| SEQ ID NO: 219 | MVVVN* |
| SEQ ID NO: 220 | MVVSN* |
| SEQ ID NO: 221 | MVAAM |
| SEQ ID NO: 222 | MVAAN* |
| SEQ ID NO: 223 | MVAVM |
| SEQ ID NO: 224 | MVAVN* |
| SEQ ID NO: 225 | MVASN* |
| SEQ ID NO: 226 | MSVAW |
| SEQ ID NO: 227 | MSVAA |
| SEQ ID NO: 228 | MSVAM |
| SEQ ID NO: 229 | MSVAN* |
| SEQ ID NO: 230 | MSVVW |
| SEQ ID NO: 231 | MSVVA |
| SEQ ID NO: 232 | MSVVM |
| SEQ ID NO: 233 | MSVVN* |
| SEQ ID NO: 234 | MSVSM |
| SEQ ID NO: 235 | MSVSN* |
| SEQ ID NO: 236 | MSAAW |
| SEQ ID NO: 237 | MSAAA |
| SEQ ID NO: 238 | MSAAM |
| SEQ ID NO: 239 | MSAAN* |
| SEQ ID NO: 240 | MSAVW |
| SEQ ID NO: 241 | MSAVA |
| SEQ ID NO: 242 | MSAVM |
| SEQ ID NO: 243 | MSAVN* |
| SEQ ID NO: 244 | MSASM |
| SEQ ID NO: 245 | MSASN* |
| SEQ ID NO: 246 | MYVAW |
| SEQ ID NO: 247 | MYVAA |
| SEQ ID NO: 248 | MYVAM |
| SEQ ID NO: 249 | MYVAN* |
| SEQ ID NO: 250 | MYVVW |
| SEQ ID NO: 251 | MYVVA |
| SEQ ID NO: 252 | MYVVM |
| SEQ ID NO: 253 | MYVVN* |
| SEQ ID NO: 254 | MYVSW |
| SEQ ID NO: 255 | MYVSA |
| SEQ ID NO: 256 | MYVSM |
| SEQ ID NO: 257 | MYVSN* |
| SEQ ID NO: 258 | MYAAW |
| SEQ ID NO: 259 | MYAAA |
| SEQ ID NO: 260 | MYAAM |
| SEQ ID NO: 261 | MYAAN* |
| SEQ ID NO: 262 | MYAVW |
| SEQ ID NO: 263 | MYAVA |
| SEQ ID NO: 264 | MYAVM |
| SEQ ID NO: 265 | MYAVN* |
| SEQ ID NO: 266 | MYASW |
| SEQ ID NO: 267 | MYASA |
| SEQ ID NO: 268 | MYASM |
| SEQ ID NO: 269 | MYASN* |
| SEQ ID NO: 281 | KLKLQ |
| SEQ ID NO: 282 | KLKQK |

^{†}X is sarcosine
^{‡}N* is norleucine

In certain embodiments related to the foregoing embodiments, the first linker and the second linker are independently selected from the structures (A), (B), (C) and (D):

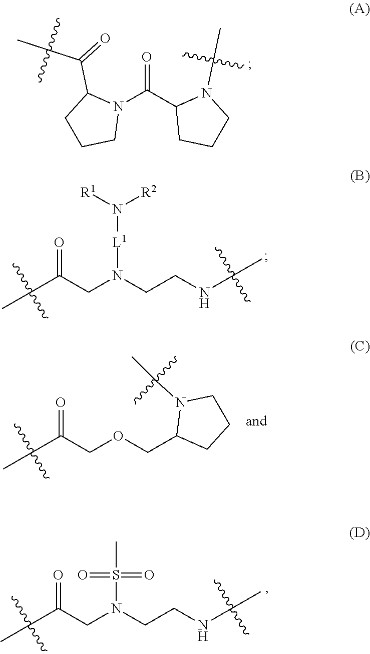

wherein:
at least one of the first and second linker is structure (B).

In other specific embodiments, the first linker is structure (B) and the second linker is structure (C). In some embodiments, the first linker is structure (B) and the second linker is structure (D).

In some embodiments, the compound has the following structure (Ia):

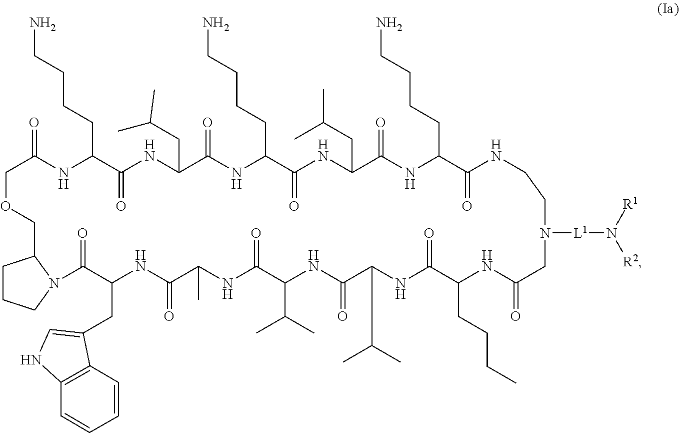

or a pharmaceutically acceptable salt, tautomer, or stereoisomer thereof.

In more specific embodiments, the compound has the following structure (Ib):

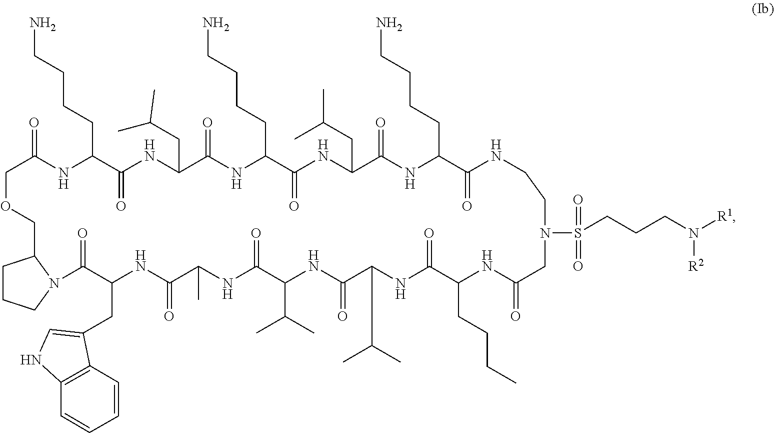

or a pharmaceutically acceptable salt, tautomer, or stereoisomer thereof.

In other more specific embodiments, the compound has the following structure:

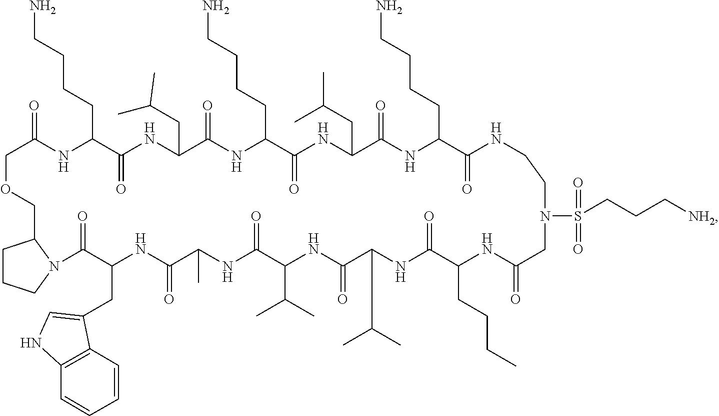 [SMILES: CCCCC1NC(=O)CN(S(=O)(=O)CCCN)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]

or a pharmaceutically acceptable salt, tautomer, or stereoisomer thereof.

In some embodiments, the compound has the following structure:

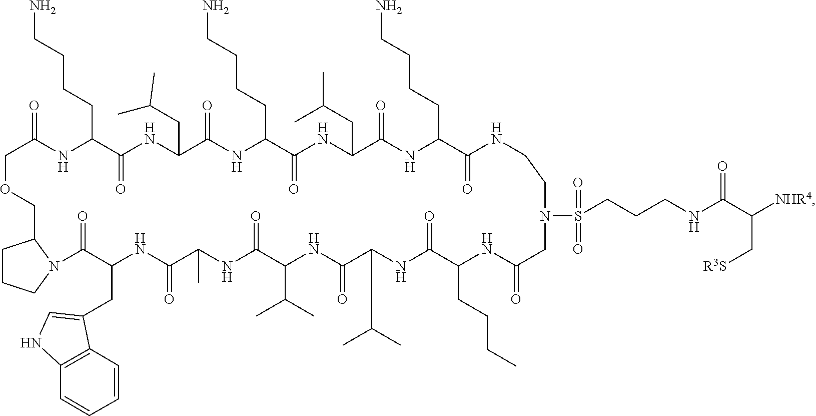

or a pharmaceutically acceptable salt, tautomer, or stereoisomer thereof.

In more specific embodiments, the compound has the following structure:

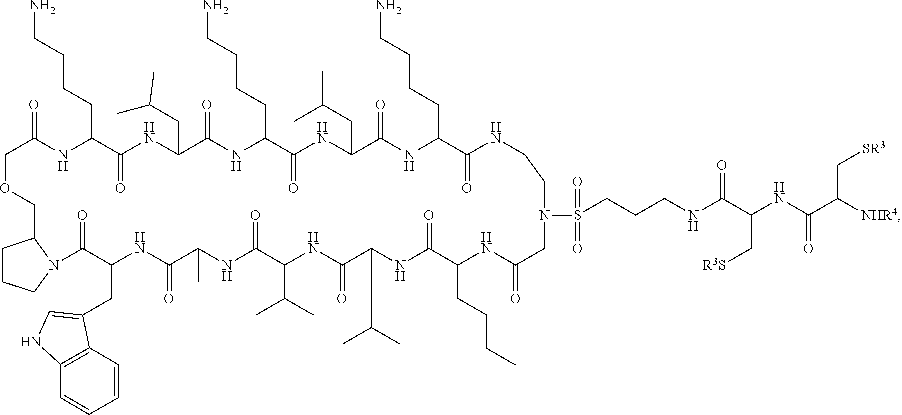

or a pharmaceutically acceptable salt, tautomer, or stereoisomer thereof.

In other embodiments, the compound has the following structure:

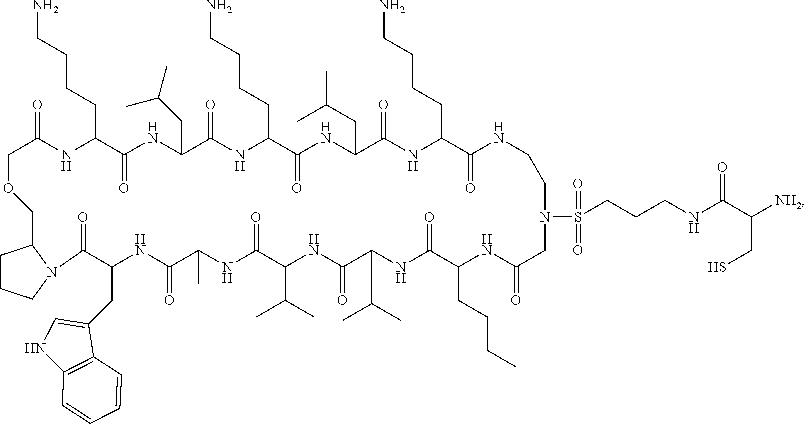 [SMILES: CCCCC1NC(=O)CN(S(=O)(=O)CCCNC(=O)C(N)CS)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]

or a pharmaceutically acceptable salt, tautomer, or stereoisomer thereof.

In certain embodiments, the compound has the following structure:

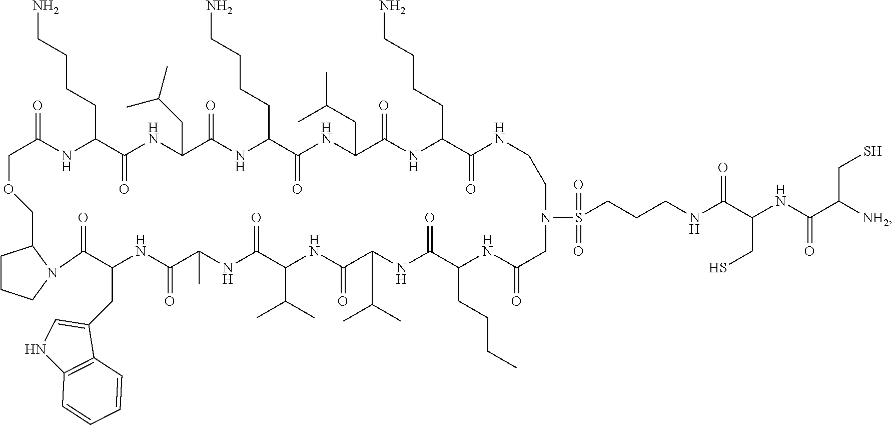 [SMILES: CCCCC1NC(=O)CN(S(=O)(=O)CCCNC(=O)C(CS)NC(=O)C(N)CS)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]

or a pharmaceutically acceptable salt, tautomer, or stereoisomer thereof.

In other more specific embodiments, the compound has the following structure (“Compound 1”):

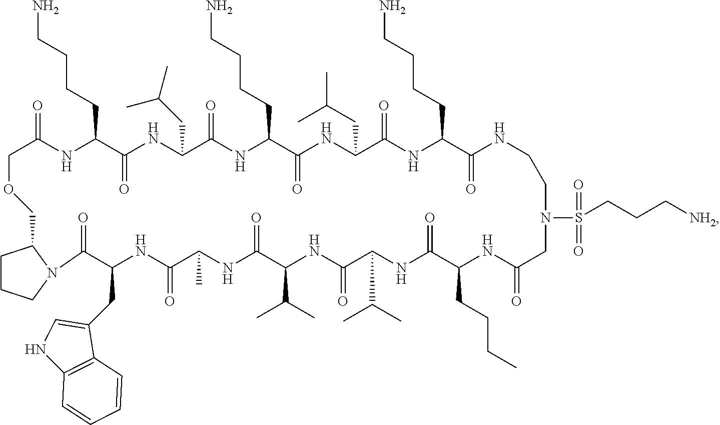 [SMILES: CCCC[C@@H]1NC(=O)CN(S(=O)(=O)CCCN)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]

or a pharmaceutically acceptable salt or tautomer thereof.

In some embodiments, the compound has the following structure (“Compound 2”):

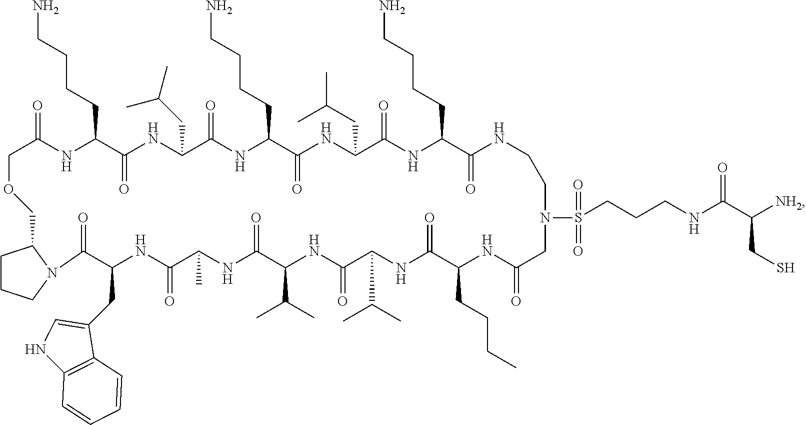 [SMILES: CCCC[C@@H]1NC(=O)CN(S(=O)(=O)CCCNC(=O)[C@@H](N)CS)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]

or a pharmaceutically acceptable salt or tautomer thereof.

In certain embodiments, the compound has the following structure (“Compound 3”):

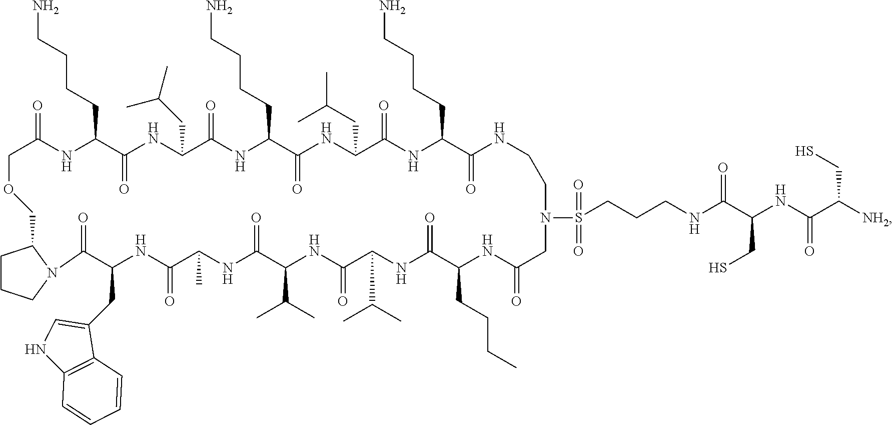 [SMILES: CCCC[C@@H]1NC(=O)CN(S(=O)(=O)CCCNC(=O)[C@H](CS)NC(=O)[C@@H](N)CS)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]

or a pharmaceutically acceptable salt or tautomer thereof.

In some embodiments, a compound is selected from a compound in Table 2, below.

TABLE 2
Exemplary compounds of structure of Formula (I)

| No. | Structure* |
|---|---|
| II-1 | 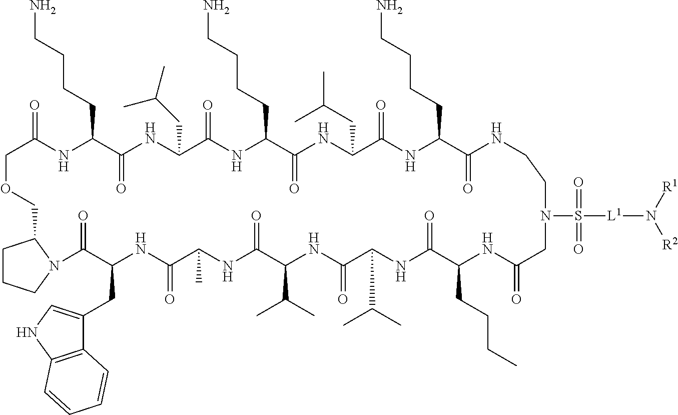 |
| II-2 | 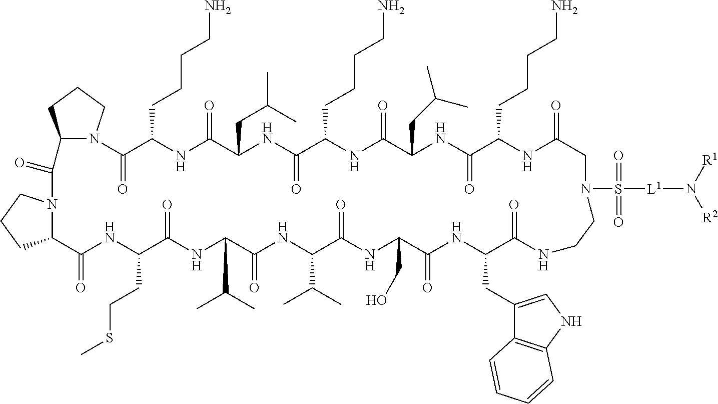 |
| II-3 | 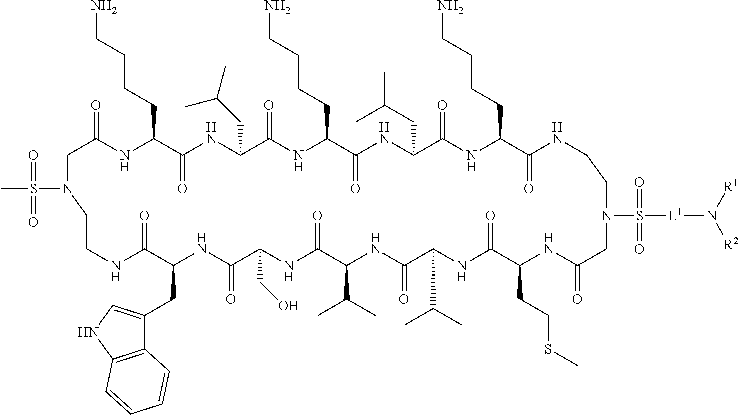 |
| II-4 | 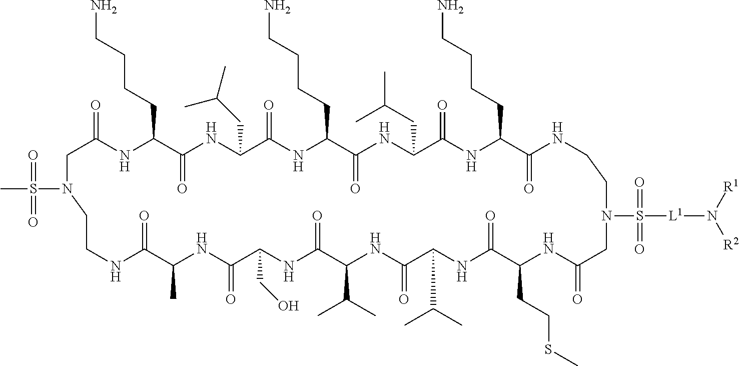 |
| II-5 | 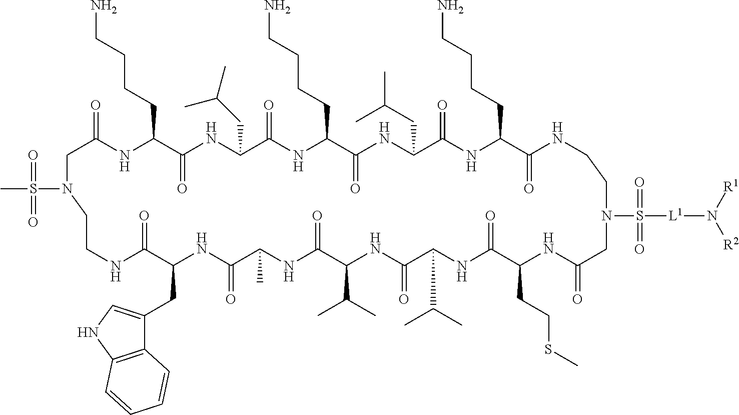 |
| II-6 | 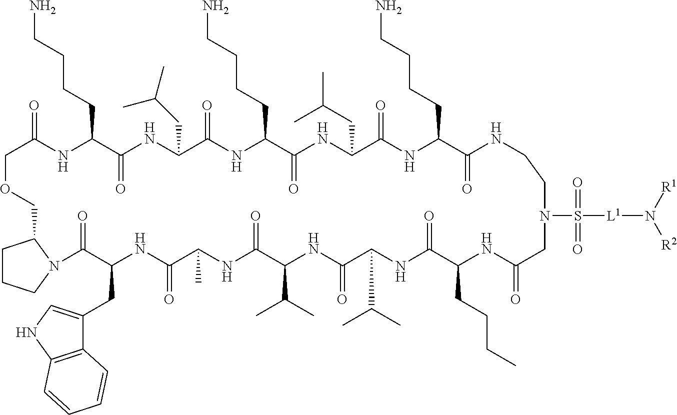 |
| II-7 | 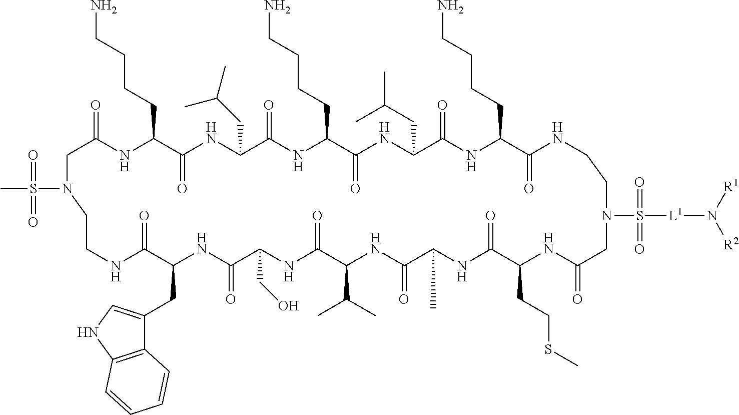 |
| II-8 | 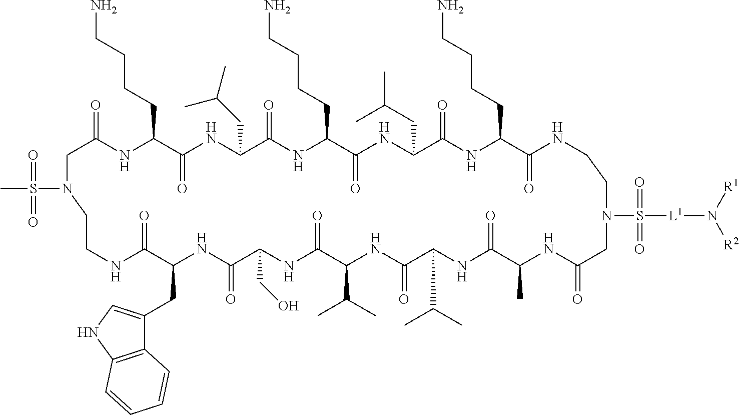 |
| II-9 | 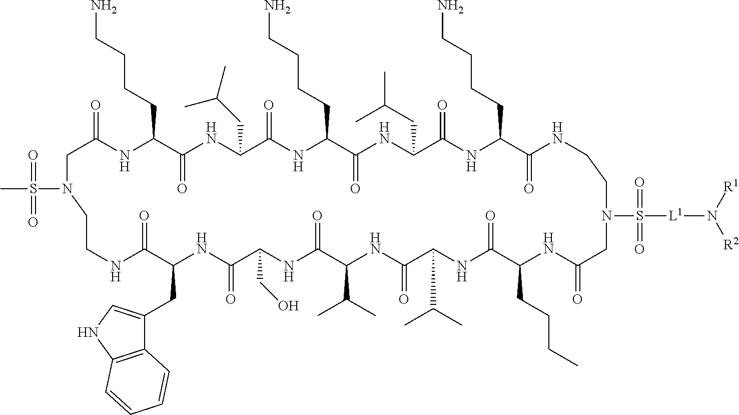 |
| II-10 | 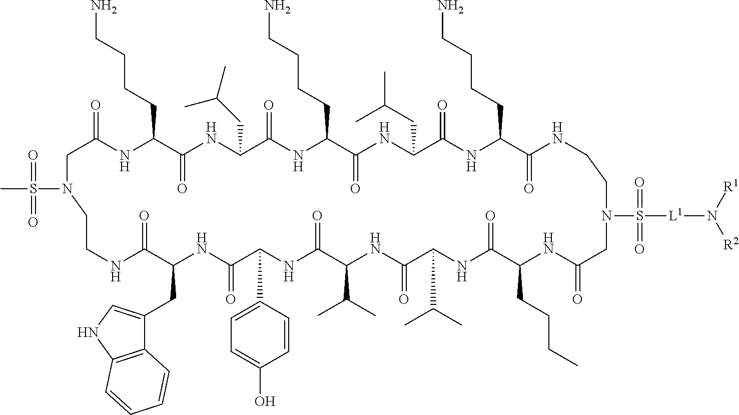 |
| II-11 | 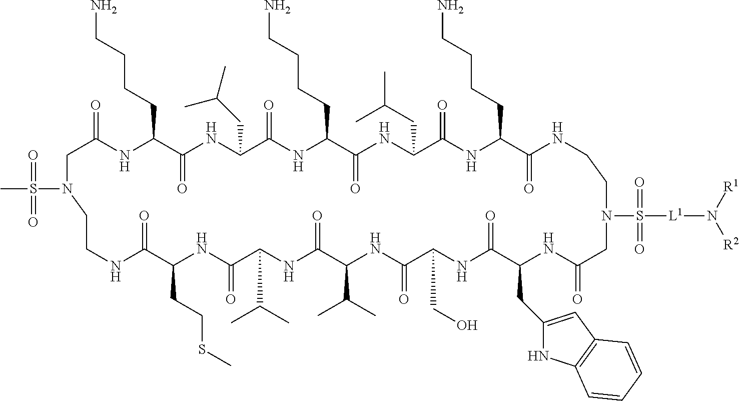 |
| II-12 | 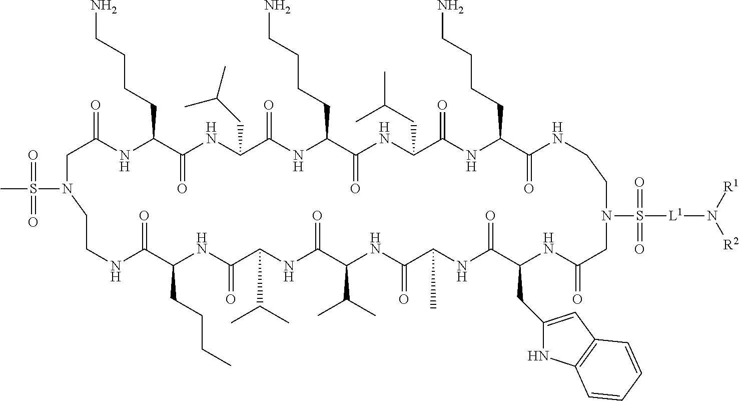 |
| II-13 | 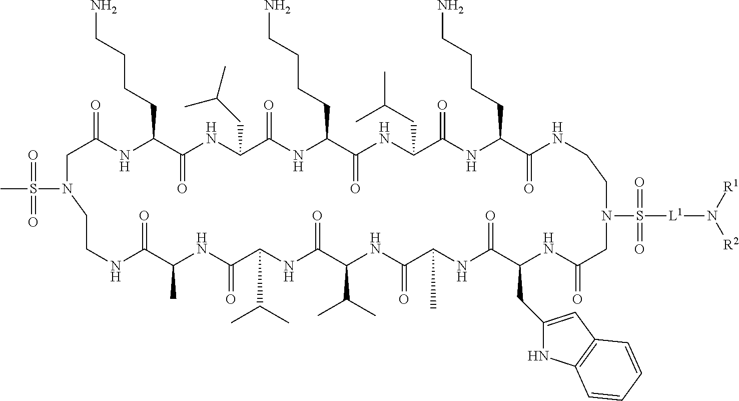 |
| II-14 | 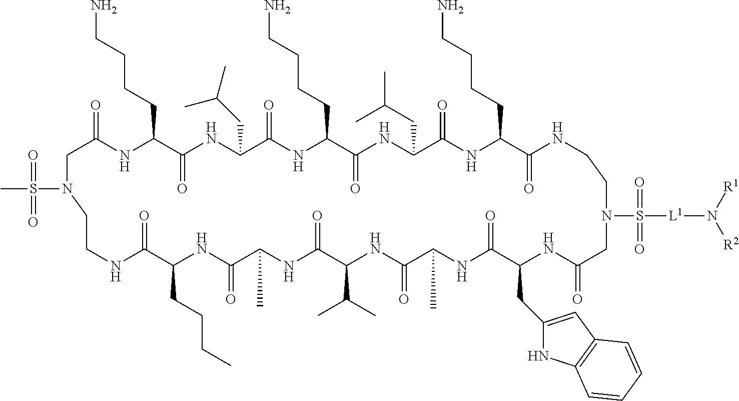 |
| II-15 | 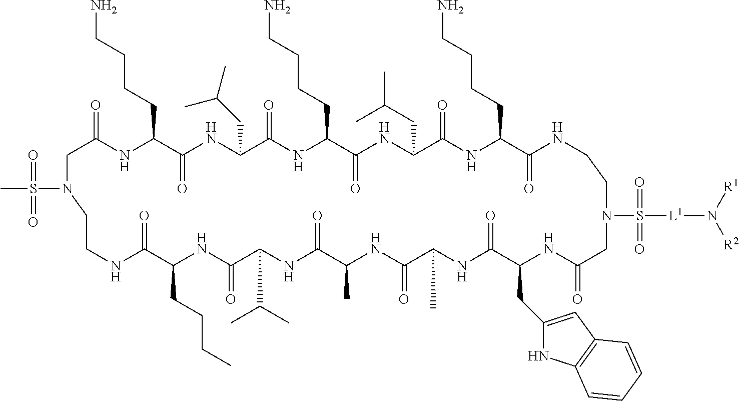 |
| II-16 | 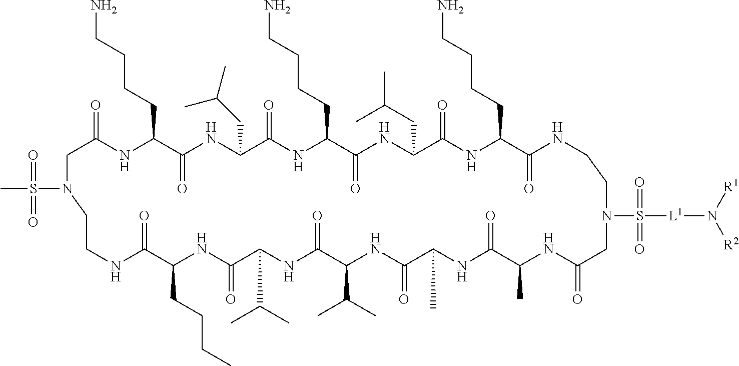 |
| II-17 | 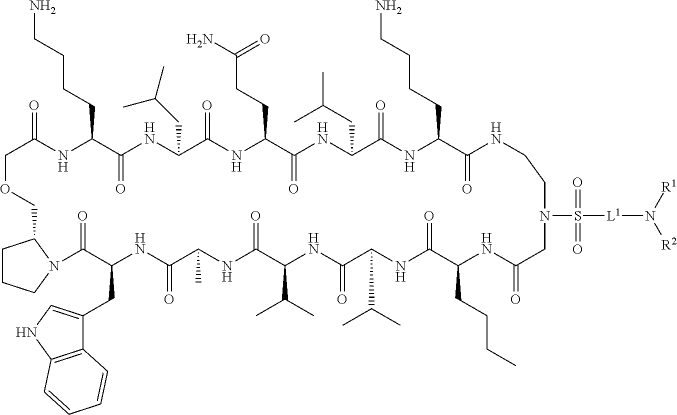 |
| II-18 | 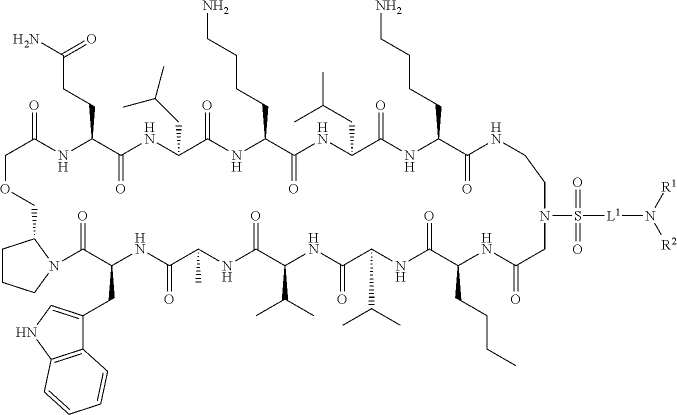 |
| II-19 | 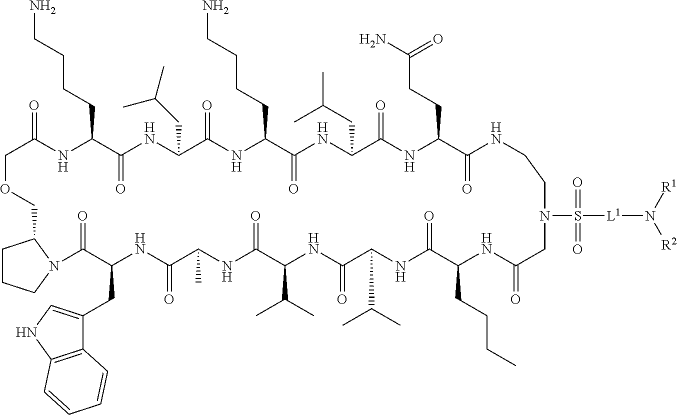 |
| II-20 | 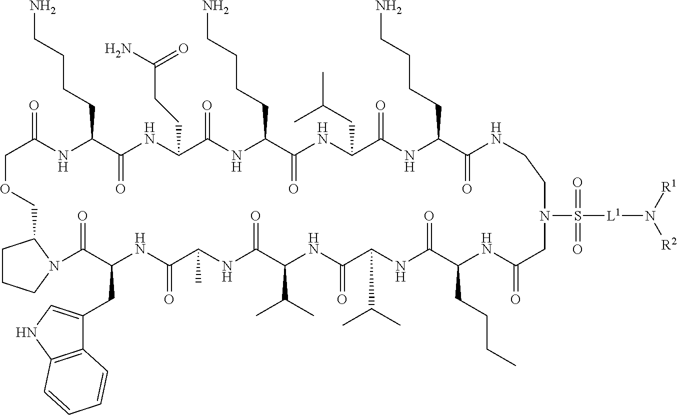 |
| II-21 | 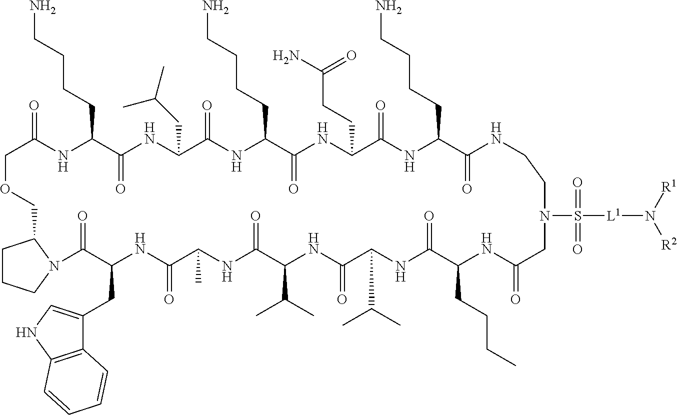 |
| II-22 | 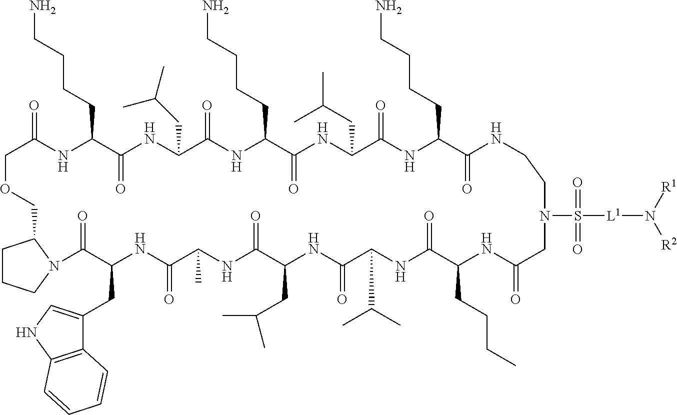 |
| II-23 | 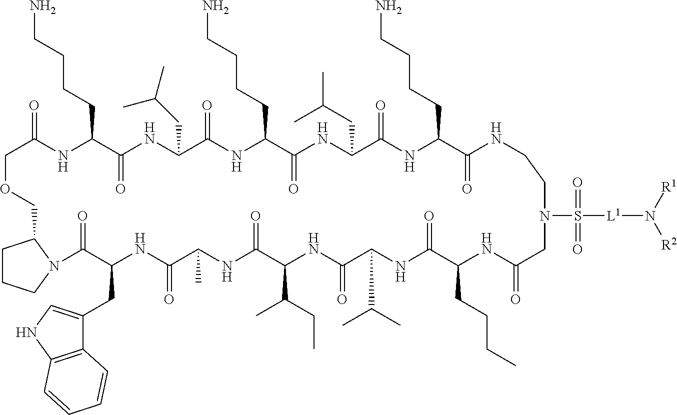 |
| II-24 | 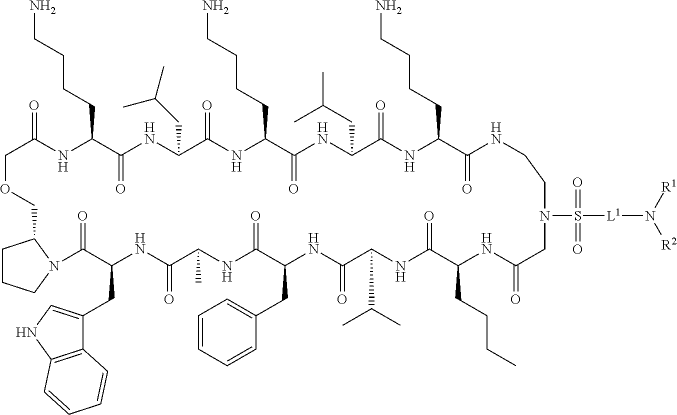 |

*L¹, R¹ and R² in Table 2 are as defined in any of the foregoing embodiments disclosed herein.

Some embodiments provide a compound selected from the compounds of Table 3 below.

TABLE 3
Exemplary compounds of structure of Formula (I)

| No. | Structure |
|---|---|
| III- 1 | 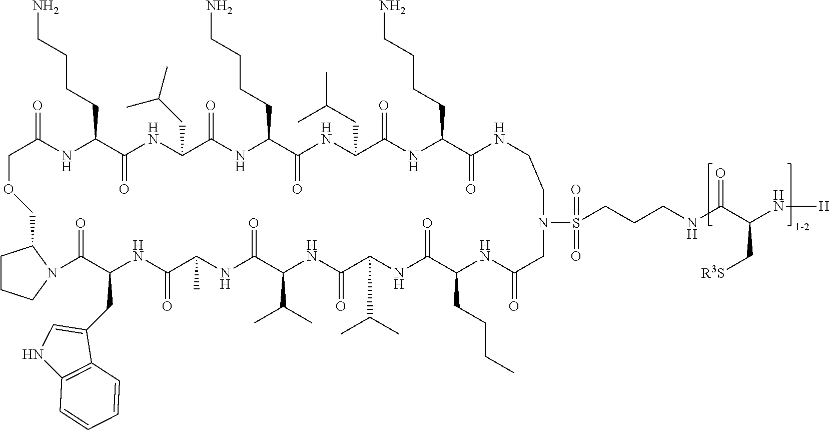 |
| III- 2 | 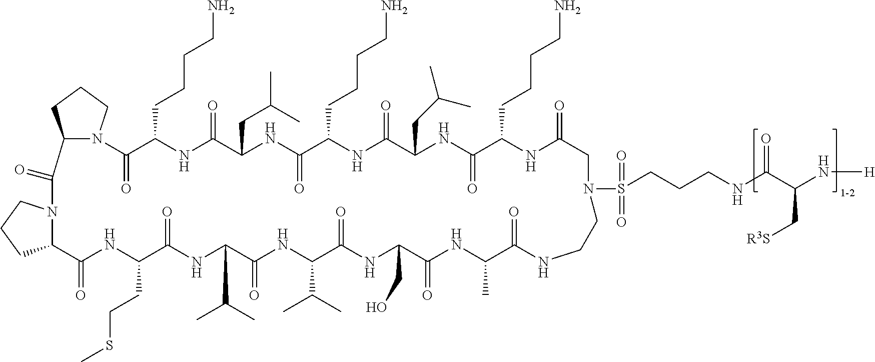 |
| III- 3 | 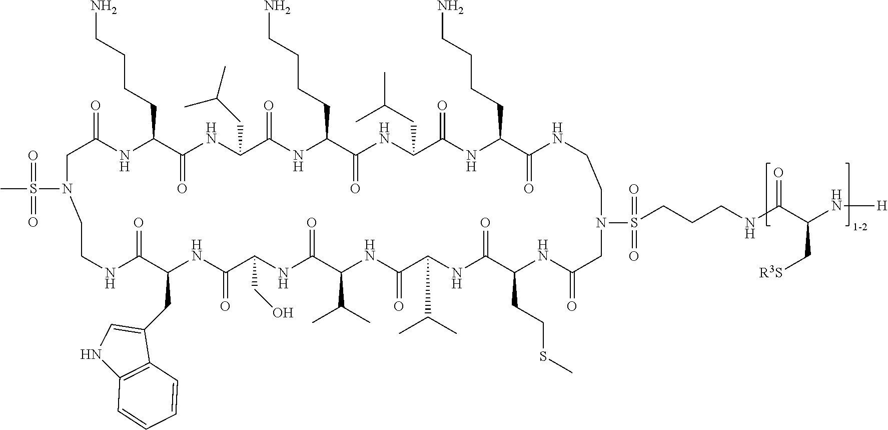 |
| III- 4 | 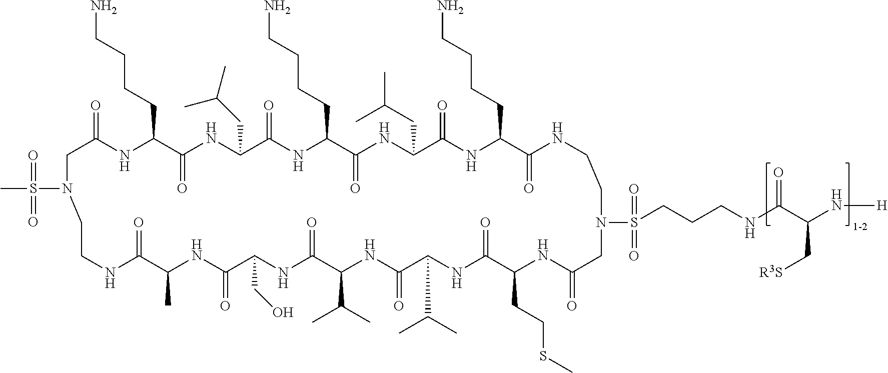 |
| III- 5 | 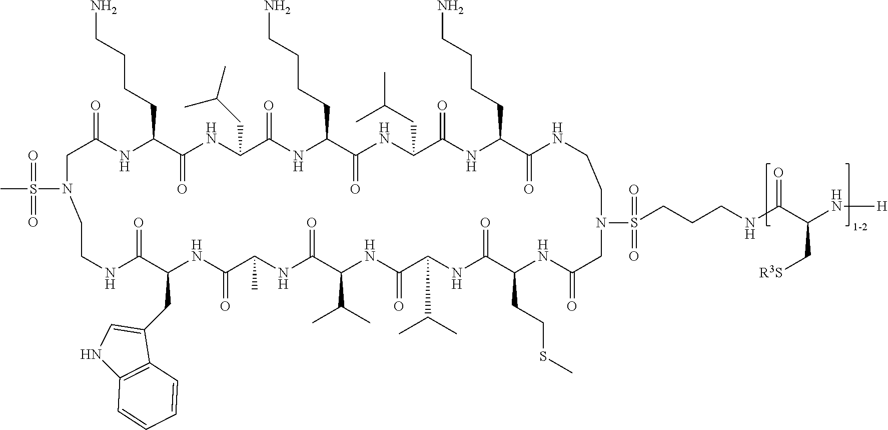 |
| III- 6 | 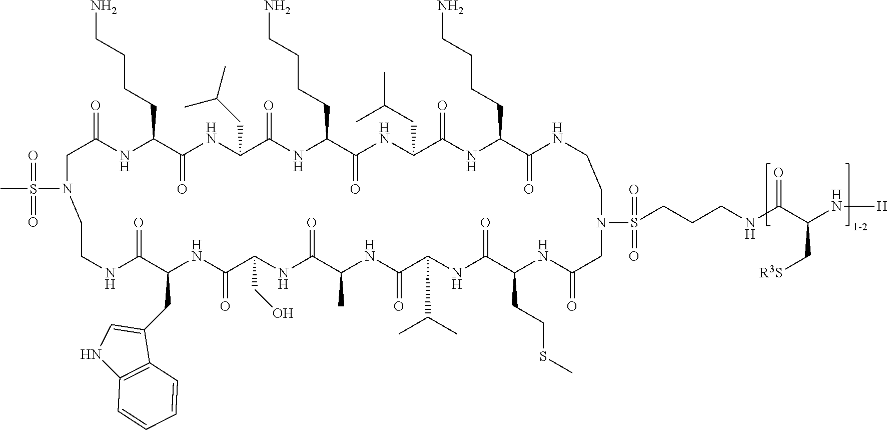 |
| III- 7 | 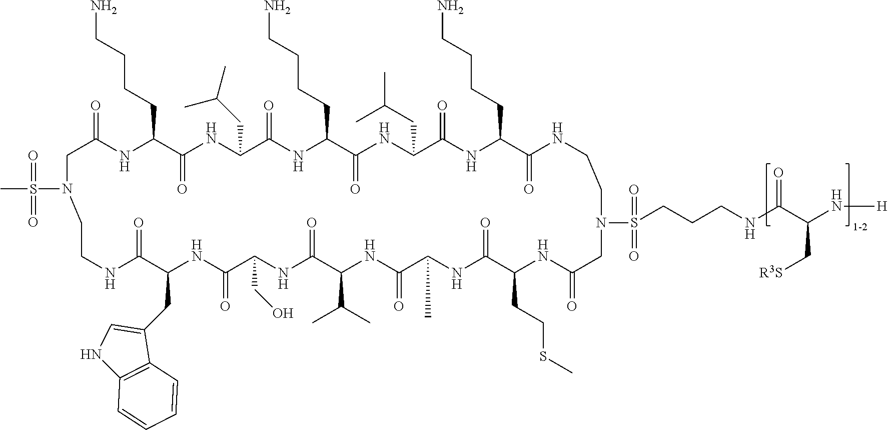 |
| III- 8 | 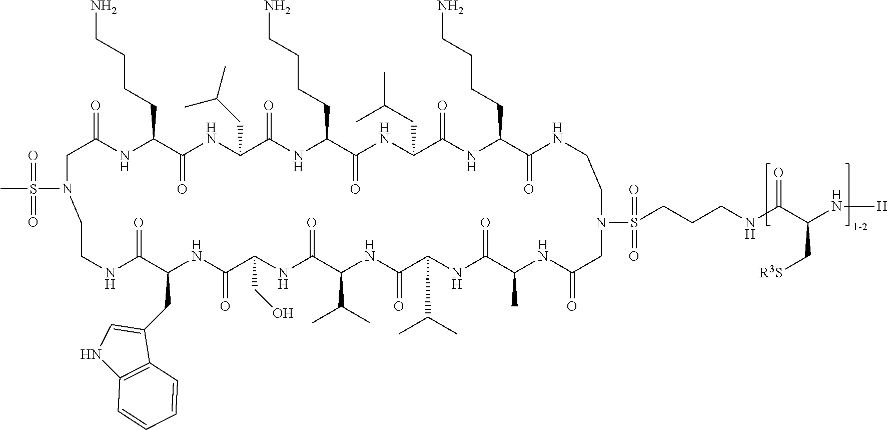 |
| III- 9 | 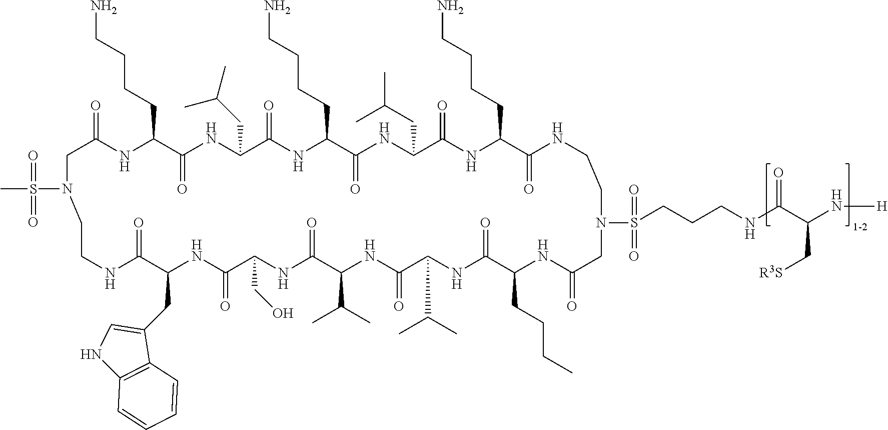 |
| III- 10 | 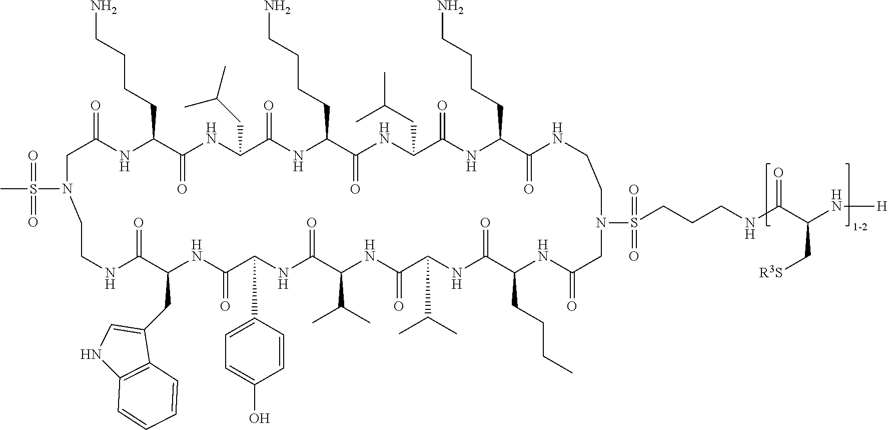 |
| III- 11 | 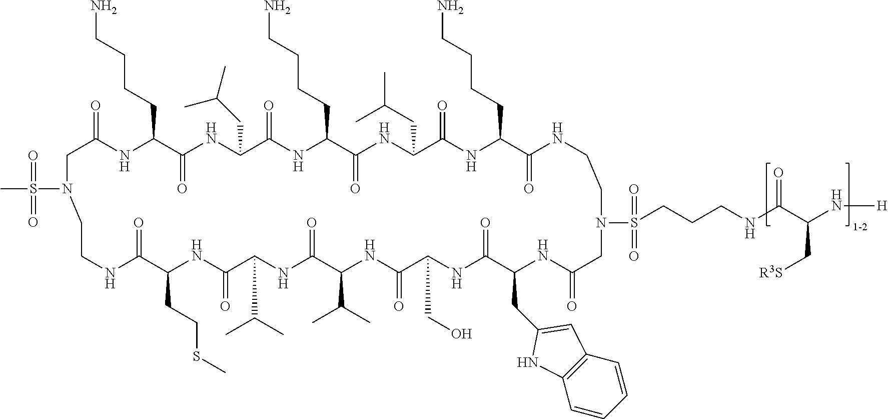 |
| III- 12 | 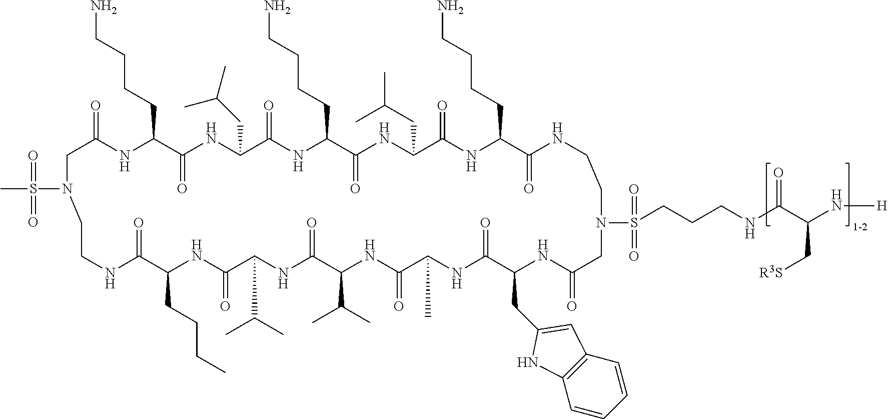 |
| III- 13 | 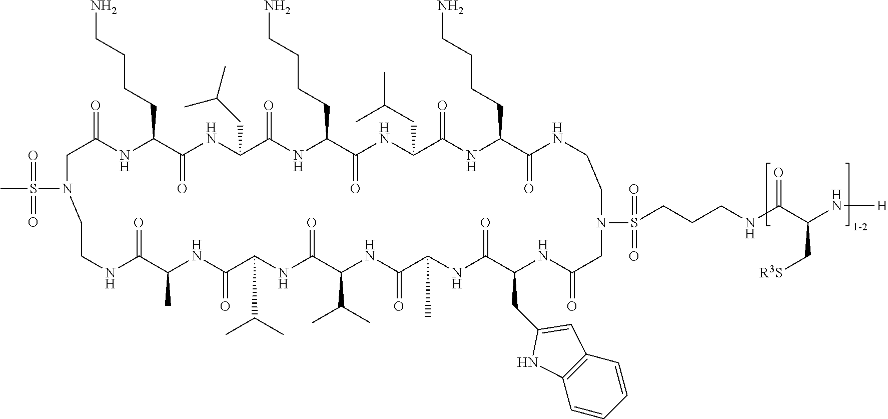 |
| III- 14 | 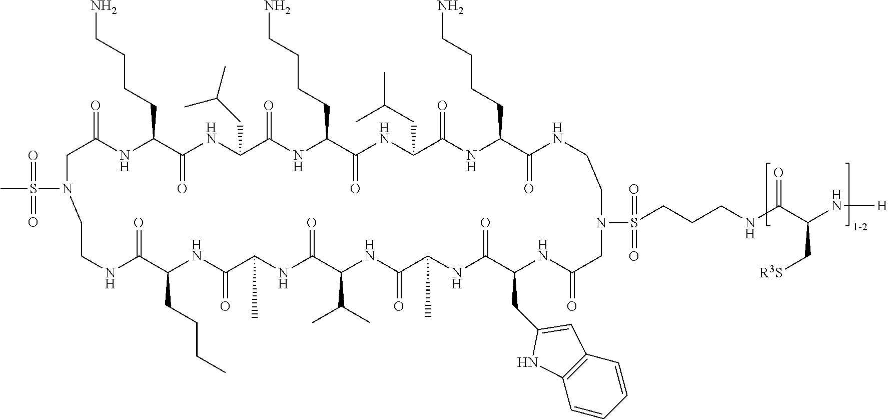 |
| III- 15 | 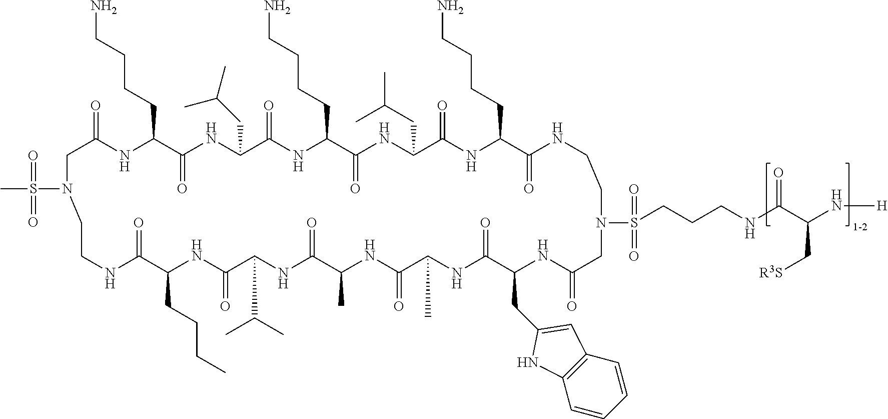 |
| III- 16 | 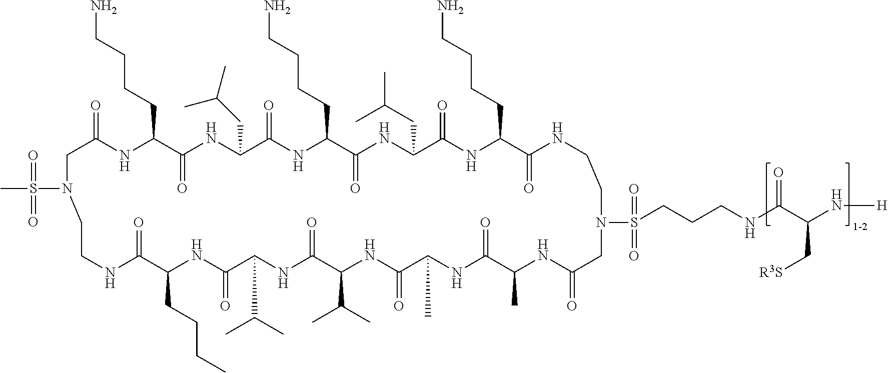 |
| III- 17 | 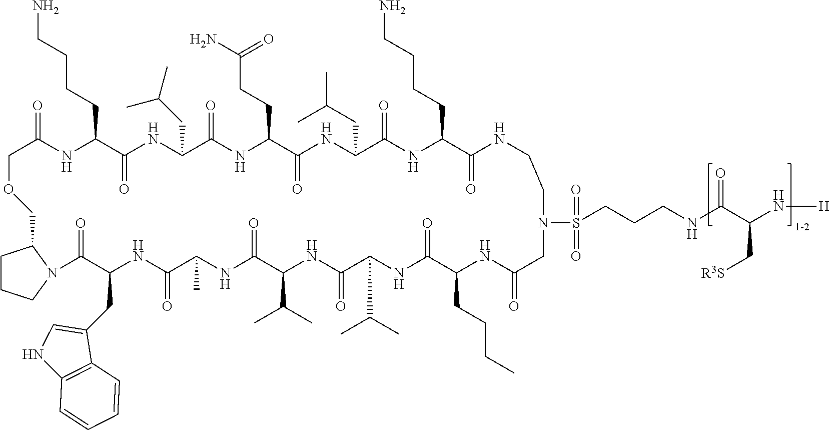 |
| III- 18 | 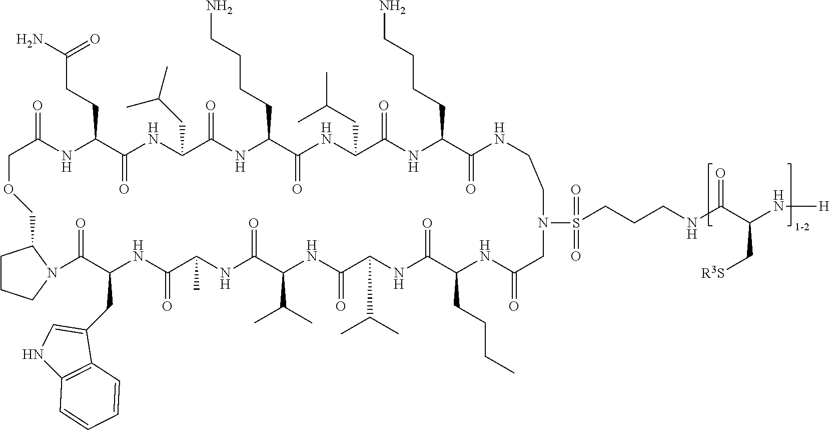 |
| III- 19 | 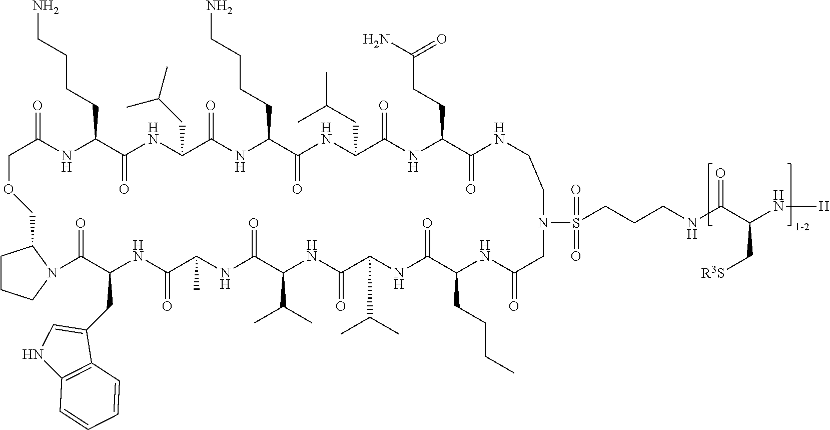 |
| III- 20 | 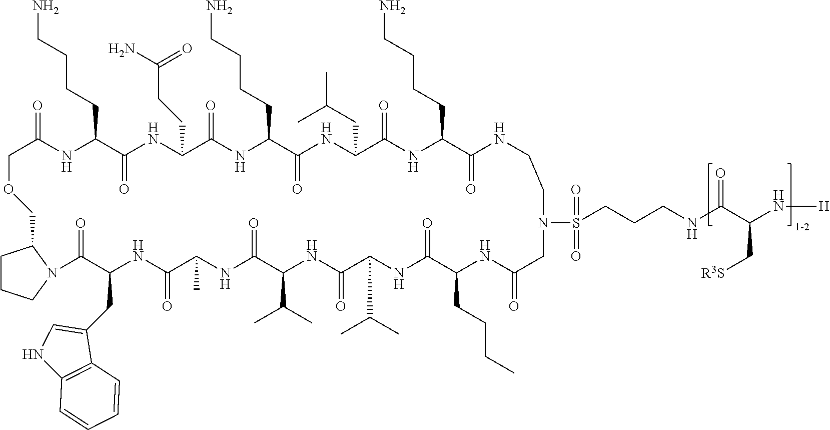 |
| III- 21 | 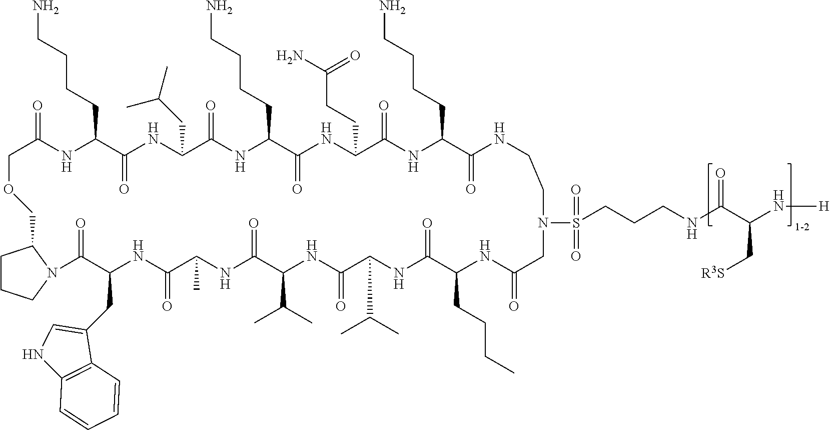 |
| III- 22 | 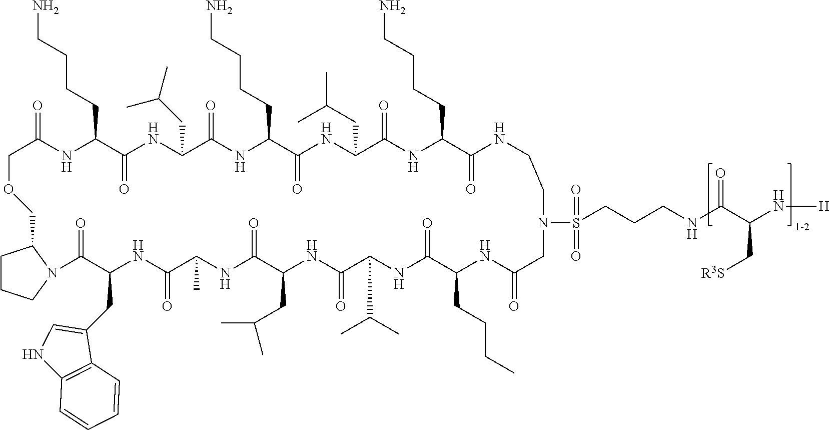 |
| III- 23 | 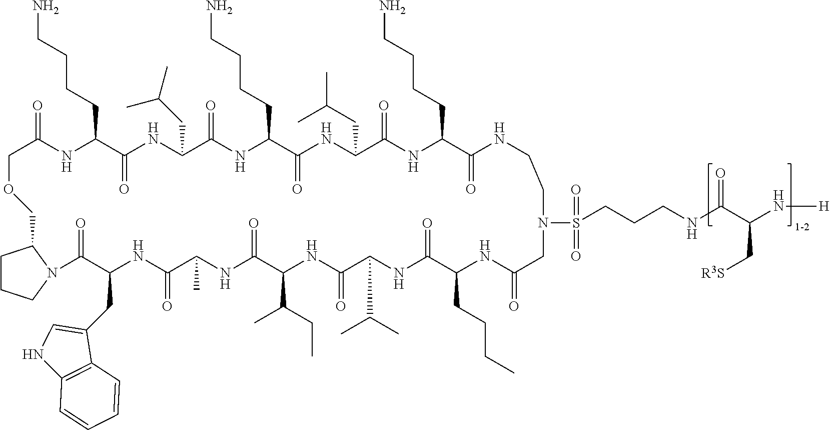 |
| III- 24 | 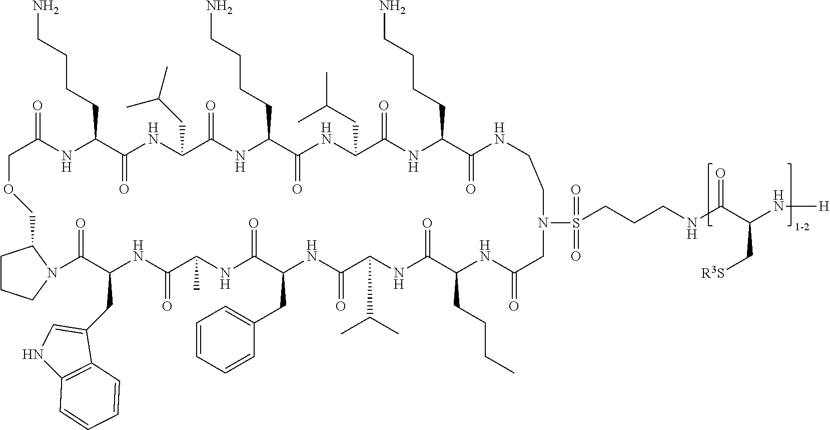 |

*Each occurrence of R³ in Table 3 is independently as defined in any of the foregoing embodiments disclosed herein.

In certain embodiments of the compounds of Table 3, the variable is 1. In other embodiments of the compounds of Table 3, the variable is 2.

### B. Compositions

In one embodiment, a composition comprising a compound according to the foregoing and a pharmaceutically acceptable carrier or excipient is described. In some embodiments, the compounds described herein (e.g., compounds of Formula (I)) are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions comprising a compound (e.g., a compound of Formula (I)) and a pharmaceutically acceptable diluent(s), excipient(s), or carrier(s) are provided herein. In certain embodiments, the compounds described are administered as pharmaceutical compositions in which compounds are mixed with other active ingredients (e.g., an anti-cancer agent), as in combination therapy. Encompassed herein are all combinations of active ingredients set forth in the combination therapies section below and throughout this disclosure. In specific embodiments, the pharmaceutical compositions include one or more compounds described herein.

A “composition” (used interchangeably with “pharmaceutical composition” herein), refers to a mixture of a compound (e.g., a compound of Formula (I)) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of a compound are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used, autoimmune responses and other factors. The compounds described herein can be used singly or in combination with one or more therapeutic agents as components of mixtures.

In one embodiment, a composition comprises a compound as described herein (e.g., a compound of Formula (I)) and a pharmaceutically acceptable carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

In some embodiments, a composition is as described above and comprises an additional bioactive agent, such as, for example, an anti-cancer agent. “Anti-cancer agent” refers to chemotherapeutic agents, cytotoxic agents, and non-peptide small molecules. Examples of anti-cancer agents include, but are not limited to Gleevec® (Imatinib Mesylate), Velcade® (bortezomib), Casodex (bicalutamide), Iressa® (gefitinib), and Adriamycin. “Anti-cancer agents” also refers to and includes alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.R™.; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included within the meaning of “anti-cancer agent” are anti-hormonal agents that act to regulate or inhibit hormone action on tumors such as anti-estrogens including for example tamoxifen (Nolvadex™), raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO). Where desired, the compounds of compositions of the present disclosure can be used in combination with commonly prescribed anti-cancer agents such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

In certain embodiments, one or more compound(s) (e.g., a compound of Formula (I)) is/are formulated in an aqueous solution. In specific embodiments, the aqueous solution is selected from, by way of example only, a physiologically compatible buffer, such as Hank's solution, Ringer's solution, or physiological saline buffer. In other embodiments, one or more compound(s) is/are formulated for transmucosal administration. In specific embodiments, transmucosal formulations include penetrants that are appropriate to the barrier to be permeated. In still other embodiments wherein the compounds described herein are formulated for other parenteral injections; appropriate formulations include aqueous or non-aqueous solutions. In specific embodiments, such solutions include physiologically compatible buffers and/or excipients.

In certain embodiments, compositions are formulated in any conventional manner using one or more physiologically acceptable carriers which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound as described herein are manufactured in a conventional manner, such as, by way of example only, by means of conventional mixing, dissolving, granulating, dragee-making, levigating, emulsifying, encapsulating, entrapping or compression processes.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the concentration of one or more compounds provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds in the pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds in the pharmaceutical composition ranges from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds in the pharmaceutical composition ranges from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

### C. Methods of Treatment

In one embodiment, a method of treating a disease, the method comprising administering to a subject in need thereof a therapeutically effective amount of a compound or composition as described herein above is presented.

The diseases that can be treated, according to compounds, compositions and methods of the present disclosure, include cancers of various types. Various cancers that can be treated, according to an embodiment of the disclosure, are well known to a person of ordinary skill in the art and such cancers are within the purview of the current disclosure.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections. Accordingly, in some embodiments, the pharmaceutical composition is formulated for oral administration. In other embodiments, the pharmaceutical composition is formulated for injection (e.g., intravenous, parenteral).

In some embodiments, a compound (e.g., a compound of Formula (I)) or composition is administered in a single dose. Typically, such administration will be by injection, e.g., intravenous injection, in order to introduce the agent quickly. However, other routes are used as appropriate. A single dose of a compound may also be used for treatment of an acute condition.

In some embodiments, the compounds or compositions is administered in multiple doses. In some embodiments, dosing is about once, twice, three times, four times, five times, six times, or more than six times per day. In other embodiments, dosing is about once a month, once every two weeks, once a week, or once every other day. In another embodiment a compound or composition and another bioactive agent are administered together about once per day to about 6 times per day. In another embodiment the administration of a compound or composition and a bioactive agent continues for less than about 7 days. In yet another embodiment the administration continues for more than about 6, 10, 14, 28 days, two months, six months, or one year. In some cases, continuous dosing is achieved and maintained as long as necessary.

Administration of the compounds (e.g., compounds of Formula (I)) and compositions may continue as long as necessary. In some embodiments, the compounds or compositions are administered for more than 1, 2, 3, 4, 5, 6, 7, 14, or 28 days. In some embodiments, the compounds or compositions are administered for less than 28, 14, 7, 6, 5, 4, 3, 2, or 1 day. In some embodiments, the compounds or compositions are administered chronically on an ongoing basis, e.g., for the treatment of chronic effects.

In some embodiments, the compounds (e.g., compounds of Formula (I)) or compositions are administered in dosages. It is known in the art that due to intersubject variability in compound or composition pharmacokinetics, individualization of dosing regimen is necessary for optimal therapy. Dosing for compounds or compositions may be found by routine experimentation in light of the instant disclosure.

In some embodiments, the disease to be treated is cancer. As such, oncological disorders within the scope of this disclosure include, but are not limited to, cancer of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain.

Specific cancers contemplated for treatment with the present disclosure include multiple myeloma, lung cancer (e.g., EGFR-driven lung cancer) and prostate cancer. Accordingly, some embodiments provide a method for treatment of multiple myeloma, the method comprising administering an effective amount of a pharmaceutical composition as disclosed herein to a subject in need thereof. Other embodiments provide a method for treatment of lung cancer (e.g., EGFR-mediated lung cancer), the method comprising administering an effective amount of a pharmaceutical composition as disclosed herein to a subject in need thereof. Still other embodiments provide a method for treatment of prostate cancer, the method comprising administering an effective amount of a pharmaceutical composition as disclosed herein to a subject in need thereof.

Other embodiments include treatment of carcinomas, Karposi's sarcoma, melanoma, mesothelioma, soft tissue sarcoma, leukemia (acute lymphoblastic, acute myeloid, chronic lymphocytic, chronic myeloid, and other), and lymphoma (Hodgkin's and non-Hodgkin's).

Other specific examples of cancers that can be treated according to the present disclosure include, but are not limited to acute lymphoblastic leukemia, acute myeloid leukemia, adrenocortical carcinoma, AIDS-related cancers (e.g., lymphoma), anal cancer, astrocytoma, basal cell carcinoma, bile duct cancer, bladder cancer, bone cancer, brain tumors (e.g., brain stem glioma, cerebellar astrocytoma, cerebral astrocytoma/malignant glioma, ependymoma, medulloblastoma, supratentorial primitive neuroectodermal, visual pathway and hypothalamic glioma), breast cancer, bronchial adenomas/carcinoids, Burkitt's Lymphoma, carcinoid tumor (e.g., gastrointestinal), cervical cancer, childhood cancers, chronic lymphocytic leukemia, chronic myelogenous leukemia, chronic myeloproliferative disorders, colon cancer, colorectal cancer, cutaneous T-cell lymphoma (e.g., mycosis fungoides and Sézary Syndrome), endometrial cancer, ependymoma, esophageal cancer, Ewing's Tumors, germ-cell tumors (e.g., extracranial, extragonadal, ovarian), extrahepatic bile duct cancer, eye cancer (e.g., intraocular melanoma, retinoblastoma), gallbladder cancer, gastric (stomach) cancer, gastrointestinal carcinoid tumors, gestational trophoblastic tumors, glioma, head and neck cancer, hepatocellular (liver) cancer, Hodgkin's Lymphoma, hypopharyngeal cancer, islet cell carcinoma, Kaposi's Sarcoma, kidney cancer (e.g., renal cell), laryngeal cancer, leukemia (e.g., hairy cell), lip and oral cavity cancer, liver cancer, lung cancer, Non-Hodgkin's Lymphoma, primary central nervous system lymphoma, Waldenström's macroglobulinemia, melanoma, mesothelioma, metastatic squamous neck cancer with occult primary, multiple endocrine neoplasia syndrome, multiple myeloma/plasma cell neoplasm, myelodysplastic syndromes, myelodysplastic/myeloproliferative diseases, nasal cavity and paranasal sinus cancer, nasopharyngeal cancer, nasopharyngeal cancer, neuroblastoma, non-small cell lung cancer, oral cancer, oropharyngeal cancer, ovarian cancer (e.g., epithelial, germ-call, low malignant potential tumor), pancreatic cancer (e.g., islet cell), paranasal sinus and nasal cavity cancer, parathyroid cancer, penile cancer, pheochromocytoma, pineoblastoma, pituitary tumors, pleuropulmonary blastoma, pregnancy cancers (e.g., breast, Hodgkin's Lymphoma, Non-Hodgkin's Lymphoma, prostate cancer, rectal cancer, renal cancer, retinoblastoma, rhabdomyosarcoma, salivary gland cancer, sarcoma (e.g., Ewing's, Kaposi's, soft tissue, uterine), skin cancer (e.g., melanoma, Merkel Cell, squamous cell carcinoma), small cell lung cancer, small intestine cancer, soft tissue sarcoma, squamous neck cancer with occult primary, supratentorial primitive neuroectodermal tumors, testicular cancer, thymoma and thymic carcinoma, thyroid cancer, transitional cell cancer of the renal pelvis and ureter, trophoblastic tumors, urethral cancer, uterine cancer (e.g., endometrial, sarcoma), vaginal cancer, vulvar cancer, and Wilms' tumors.

In certain embodiments of the method described above, the method further comprises administering a bioactive agent to the subject simultaneously or consecutively with the compound or composition. In some more specific embodiments, the bioactive agent is an anti-cancer agent. In some of the foregoing embodiments, the bioactive agent is administered within the same formulation as the compound. In other embodiments, the bioactive agent is administered within a formulation that is separate from the compound.

In one embodiment, a method for delivering a compound to a cell or tissue, the method comprising administering to the cell or tissue in vitro or in vivo a compound or composition as described herein above is presented. In some of those embodiments, the cell or tissue is diseased, for example, a cancer cell or tissue.

### D. Methods of Preparation

One embodiment provides a method for preparing a compound having a structure of Formula (I):

X-L¹-N(R¹)R², (I)

or a pharmaceutically acceptable salt, tautomer, or stereoisomer thereof, wherein:
X is a cyclic β-hairpin peptidomimetic;
L¹ is an optional linker; and
R¹ is H; and
R² is H or a moiety comprising one or more amino acid residues or substituted derivatives thereof. For example, the compound may be a compound having a structure of Formula (I) according to any of the embodiments described herein. Accordingly, in some embodiments, the method comprises:

One embodiment provides a method for preparing a compound having a structure of Formula (I), the method comprising modifying a cyclic β-hairpin peptidomimetic to include a linker L¹. In some more specific embodiments, the linker L¹ is modified to include one or more amino acid residues or derivatives thereof.

In certain specific embodiments, the linker L¹ is modified to include one of the following structures:

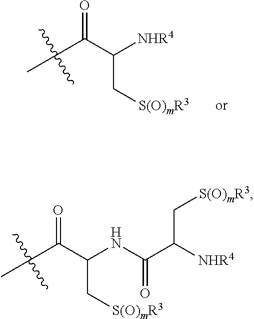

wherein:
R³ is, at each occurrence, independently H, a protecting group, alkyl, aminoalkyl, a protected aminoalkyl, hydroxyalkyl, a protected hydroxyalkyl, or -L²-Y;
R⁴ is H or an amine protecting group;
m is, at each occurrence, independently, 0, 1 or 2;
L² is a linker; and
Y at each occurrence independently comprises one or more saccharide moieties.

Embodiments of the methods for preparing a compound having a structure of Formula (I) may include one more amino acid coupling steps according to the following Amino Acid Coupling Reaction Scheme.

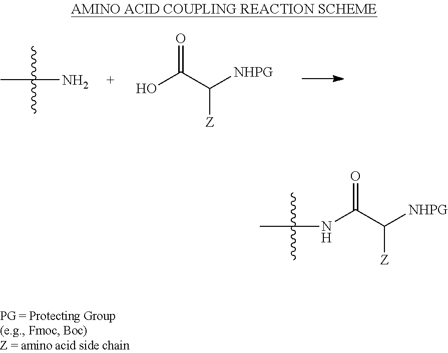

As shown above, the [symbol] may represent an attachment to an additional amino acid residue, a solid support, or linker (e.g., L¹). The protecting group can be selected based on the overall synthetic scheme (e.g., accounting for protecting groups of side chains, etc.). In one particular embodiment, the method for preparing a compound having a structure of Formula (I) includes an amino acid coupling step according to the amino acid coupling reaction scheme above. In more specific embodiments, the protecting group is an Fmoc protecting group.

In some embodiments, the amino acid side chain (“Z”) is a cysteine side chain. In some embodiments, the amino acid side chain is a protected form of the amino acid side chain. In some embodiments, the amino acid side chain comprises H, a protecting group, alkyl, aminoalkyl, a protected aminoalkyl, hydroxyalkyl, a protected hydroxyalkyl, or -L²-Y, wherein L² and Y are defined according to the embodiments described herein.

The examples and preparations provided below further illustrate and exemplify the compounds and compositions of the present disclosure as well as methods of preparing and using such compounds. It is to be understood that the scope of the present disclosure is not limited in any way by the scope of the following examples and preparations.

### EXAMPLES

### Example 1

### Synthesis of Exemplary Compound of Formula (I) (Compound 2)

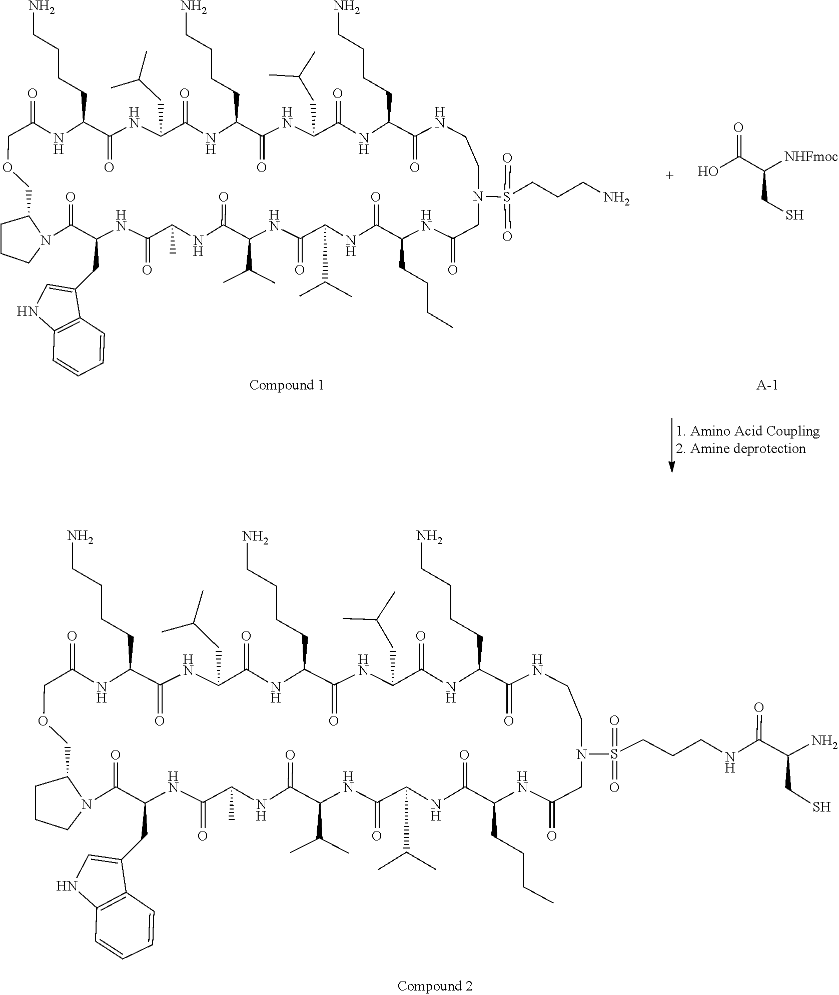

Compound 1 was obtained using standard peptide synthesis techniques (e.g., solid phase synthesis). Compound 1 was then coupled to the Fmoc-protected cysteine amino acid (“A-1”) using standard amino acid coupling conditions (e.g., using carbodiimides such as DCC, DIC, or EDC, or alternatively, with aminium/uronium and phosphonium salts). The resultant compound was deprotected under appropriate conditions (e.g., 20-50% piperidine in DMF) to afford the desired product, which can be purified using standard techniques (e.g., silica gel or HPLC preparative chromatography). Alternatively, additional amino acid coupling steps can be used following the deprotection of the amine to add amino acid residues as desired (e.g., to obtain Compound 3).

### Example 2

### Alkylation of Cysteine Derivatives

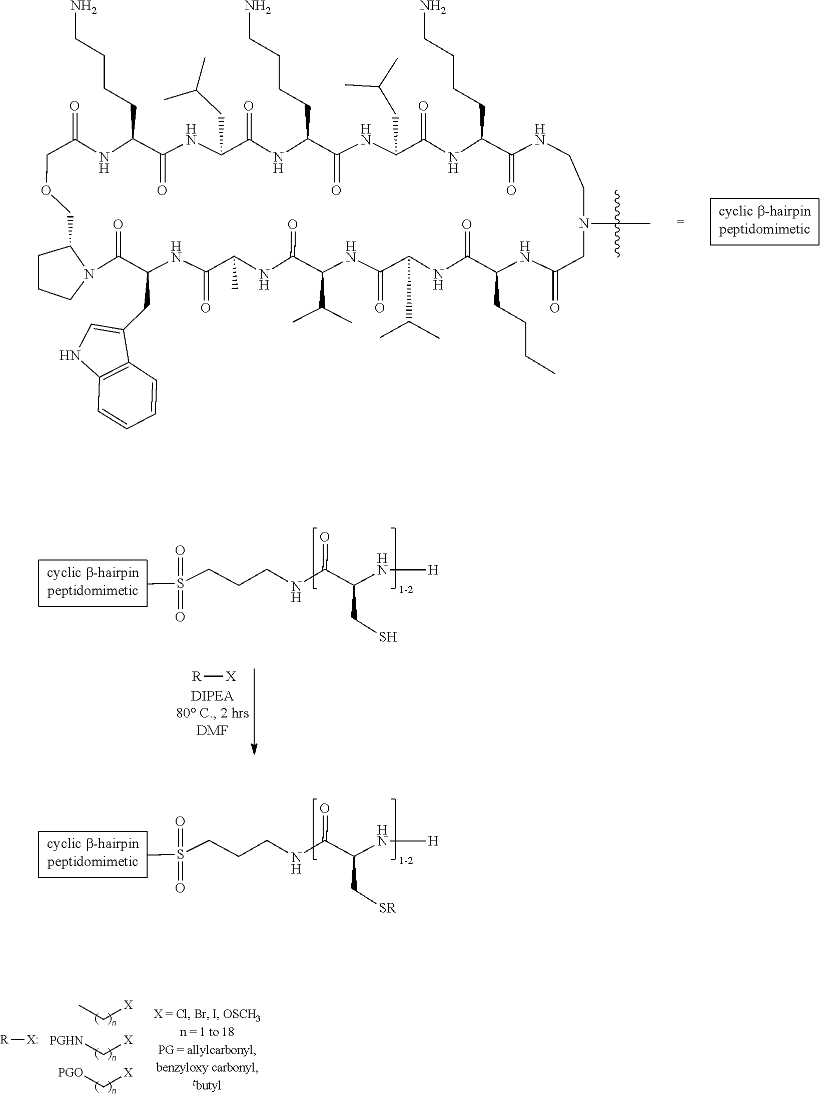

Cysteine derivatives of the exemplary compounds comprising a cyclic β-hairpin peptidomimetic synthesized according to Example 1 (e.g., Compound 2 or Compound 3; 1 equivalent) were dissolved in DMF with the desired alkylating agent (“R-X”; 1.05 equivalents). Diisopropylethylamine (2.2 equivalents) was added to the reaction mixture and the mixture was heated to 80° C. The reaction was monitored by thin layer chromatography using iodine stain and after the reaction is judged to be complete (approximately 2 hours) solvents were removed in vacuo. Protecting groups were removed using standard techniques (e.g., concentrated TFA at room temperature). The crude desired product was then purified by reverse phase chromatography to afford pure compounds. Also, saccharides shown below can be added selectively via the alkylation of the cysteine thiol groups. Alternatively the saccharides can added before the cysteines are attached to the cyclic β-hairpin peptidomimetic as shown in Example 2 below.

### Example 3

### Synthesis of Saccharide Derivatives

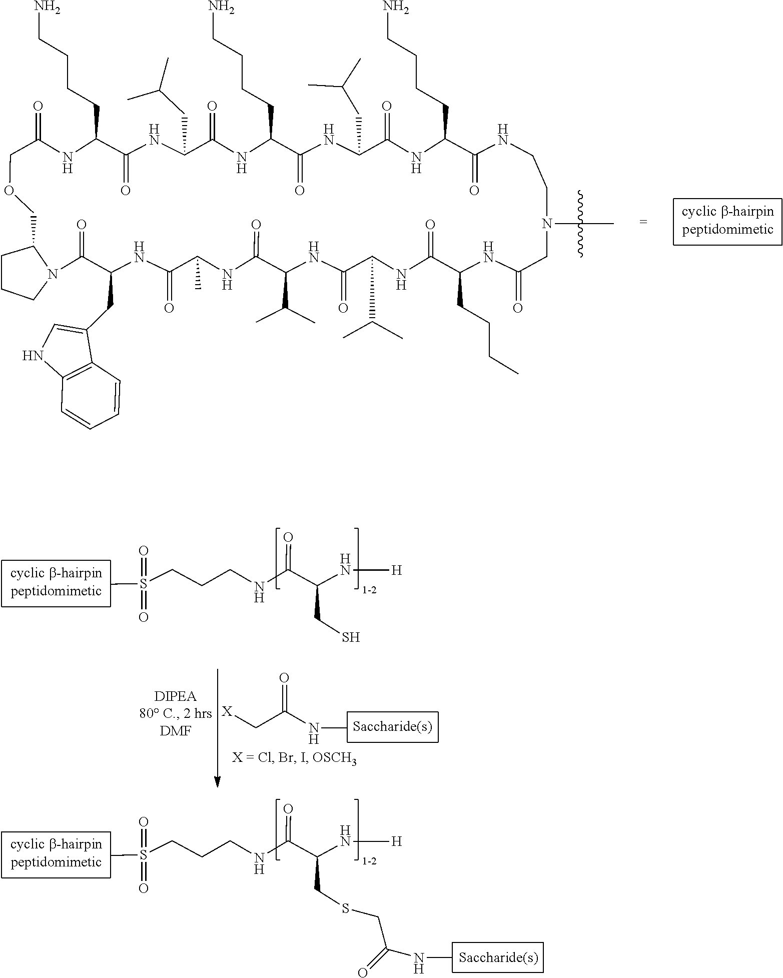

Cysteine derivatives of the exemplary compounds comprising a cyclic β-hairpin peptidomimetic synthesized according to Example 1 (e.g., Compound 2 or Compound 3; 1 equivalent) were dissolved in DMF with the desired saccharide derivative (1.05 equivalents). Diisopropylethylamine (2.2 equivalents) was added to the reaction mixture and the mixture was heated to 80° C. The reaction was monitored by thin layer chromatography using iodine stain and after the reaction is judged to be complete (approximately 2 hours) solvents were removed in vacuo. The crude desired product was then purified by reverse phase chromatography to afford pure compounds.

### Example 4

### Cysteine Derivatives

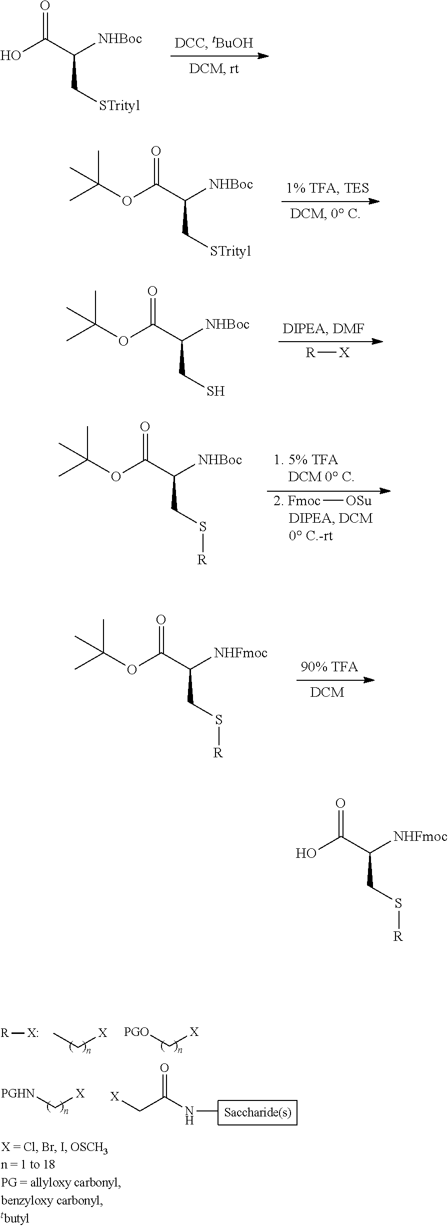

As an alternative to the synthetic strategies described in Examples 2 and 3, a cysteine amino acid monomer unit can be alkylated or derivatized with a saccharide derivative prior to attachment to the free amine of Compound 1. Using this strategy, the alkylating agents or saccharide derivatives attached to the sulfur of the cysteine residue can be the same or different and can be selected to modulate biological activity, solubility and bioavailability as desired.

### Example 5

### Potency of Exemplary Compounds

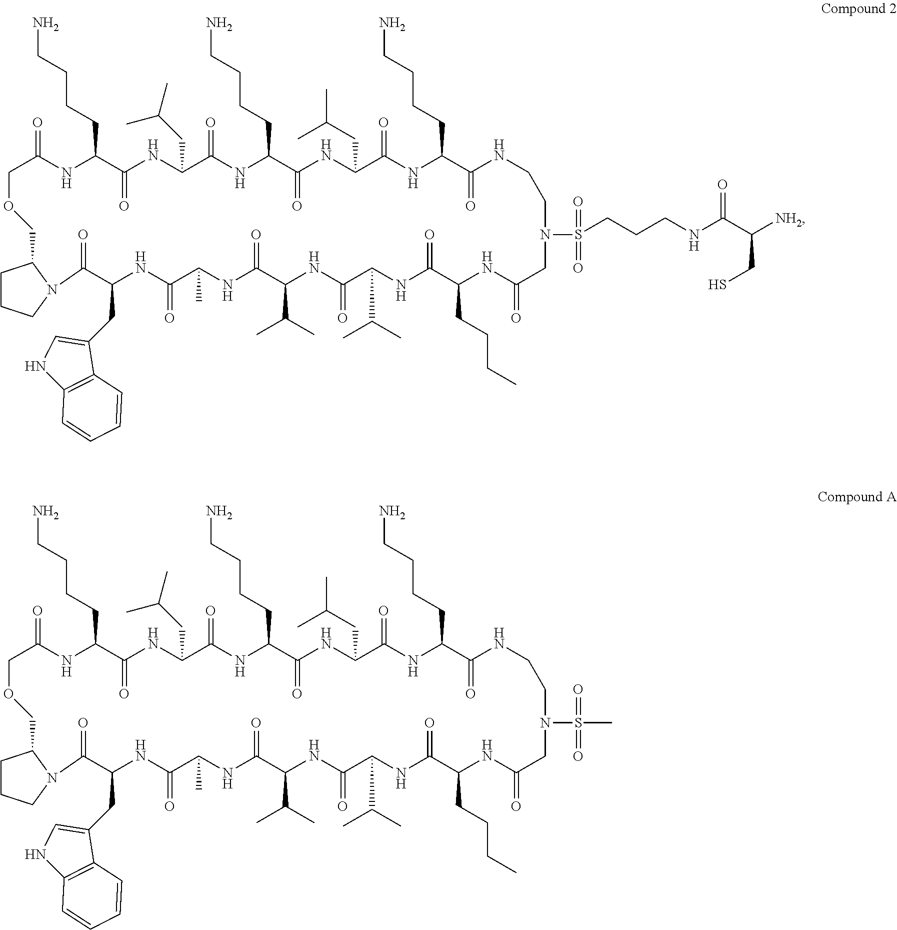 [SMILES: CCCC[C@@H]1NC(=O)CN(S(=O)(=O)CCCNC(=O)[C@@H](N)CS)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O ; CCCC[C@@H]1NC(=O)CN(S(C)(=O)=O)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]

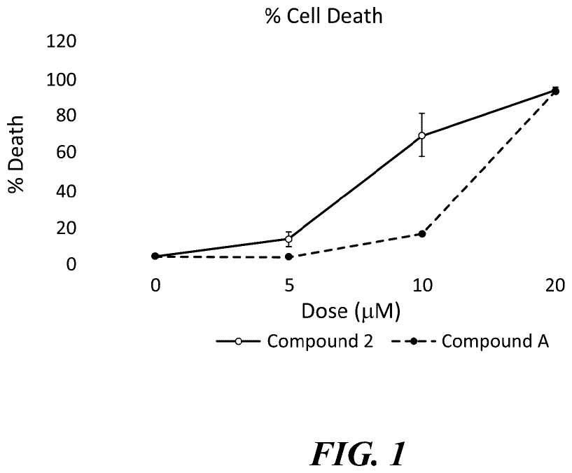

An exemplary embodiment of the compounds disclosed herein (“Compound 2”) shows increased potency relative to another known cyclic β-hairpin peptidomimetic compound (“Compound A”). Compound 2 was tested alongside Compound A and cell death was measured in a U266 myeloma cell line. Dapi staining was used for detection of dead cells and Fluo-4 was used for staining live cells. Cells were then imaged at 6 hours using a Cytation 5 Biotek imaging plate reader. Compound 2 showed more potency compared to Compound A, with results reported as the mean and error bars indicating standard deviation (see FIG. 1, n=3).

The preparation of the cysteine derivatives described in Example 4 can be performed according to or adapted from the protocols described in Yuko Tsuda, Chem. Pharm. Bull. 1991, 39, 607-611 which is hereby incorporated by reference in its entirety. Additionally, methods for synthesizing the compounds comprising cyclic β-hairpin peptidomimetics (e.g., Compound 1, Compound 2 and Compound 3) and derivatives thereof as described herein are known in the art (i.e., peptide synthesis), for example as described in U.S. Pat. No. 8,853,149 and U.S. Pub. No. 2014/0322227 which are hereby incorporated by reference in their entirety.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/680,904, filed Jun. 5, 2018, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-55. (canceled)

56. A method for treatment of cancer, the method comprising administering an effective amount of a compound having the following structure: 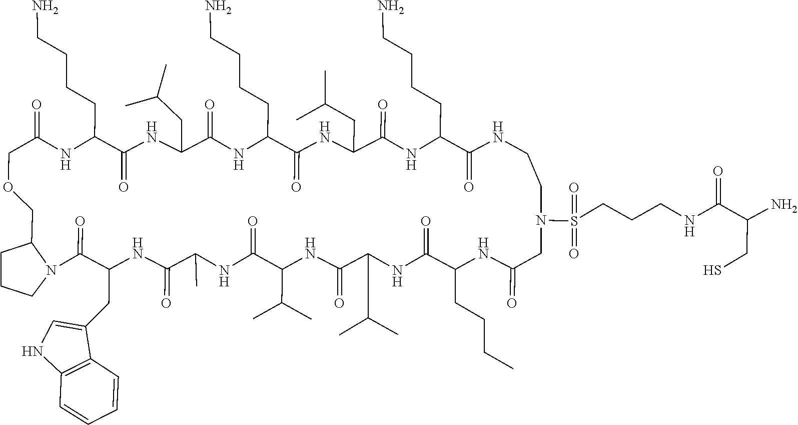 [SMILES: CCCCC1NC(=O)CN(S(=O)(=O)CCCNC(=O)C(N)CS)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]
or a pharmaceutically acceptable salt, tautomer, or stereoisomer thereof, to a subject in need thereof.

57. The method of claim 56, wherein the cancer is multiple myeloma.

58. The method of claim 56, wherein the cancer is lung cancer.

59. The method of claim 58, wherein the lung cancer is EGFR-mediated lung cancer.

60. The method of claim 56, wherein the cancer is prostate cancer.

61. The method of claim 56, wherein the compound is formulated with a pharmaceutically acceptable carrier or excipient.

62. The method of claim 56, wherein the compound is administered orally.

63. The method of claim 56, wherein the compound is administered via an injection.

64. The method of claim 63, wherein the injection is an intravenous injection.

65. The method of claim 56, wherein the compound is administered via pulmonary or nasal administration.

66. The method of claim 56, wherein the compound has the following structure: 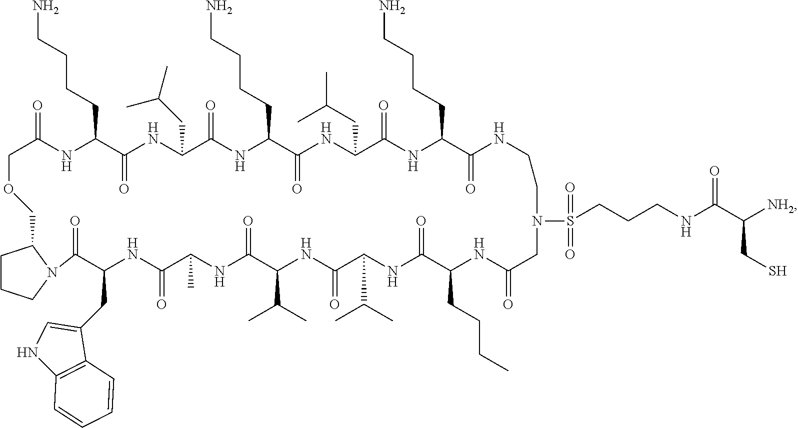 [SMILES: CCCC[C@@H]1NC(=O)CN(S(=O)(=O)CCCNC(=O)[C@@H](N)CS)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]
or a pharmaceutically acceptable salt or tautomer thereof.

67. The method of claim 56, wherein the administration is a single dose.

68. The method of claim 56, wherein the administration is multiple doses.

69. The method of claim 56, wherein the subject is a mammal.

70. The method of claim 67, wherein the mammal is a human.

71. A compound having the following structure: 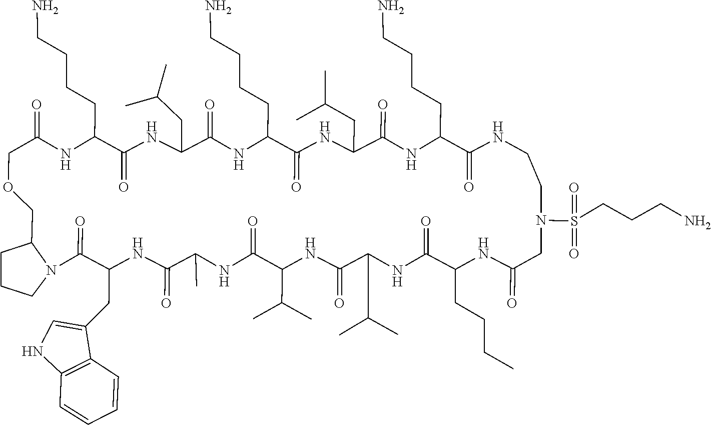 [SMILES: CCCCC1NC(=O)CN(S(=O)(=O)CCCN)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]
or pharmaceutically acceptable salt, tautomer, or stereoisomer thereof.

72. The compound of claim 69, wherein the compound has the following structure: 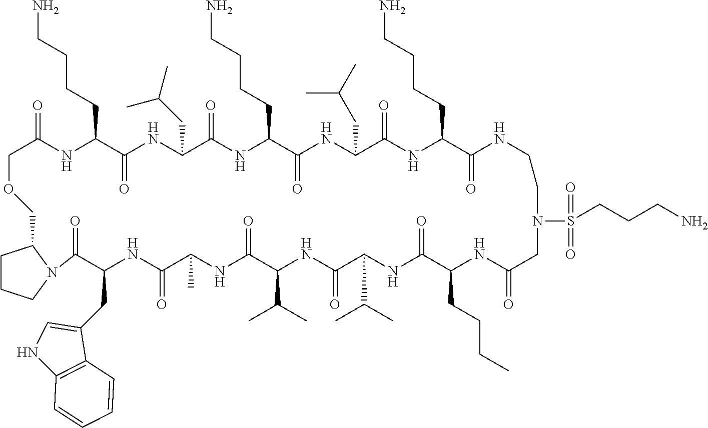 [SMILES: CCCC[C@@H]1NC(=O)CN(S(=O)(=O)CCCN)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]
or a pharmaceutically acceptable salt or tautomer thereof.

73. A compound having the following structure: 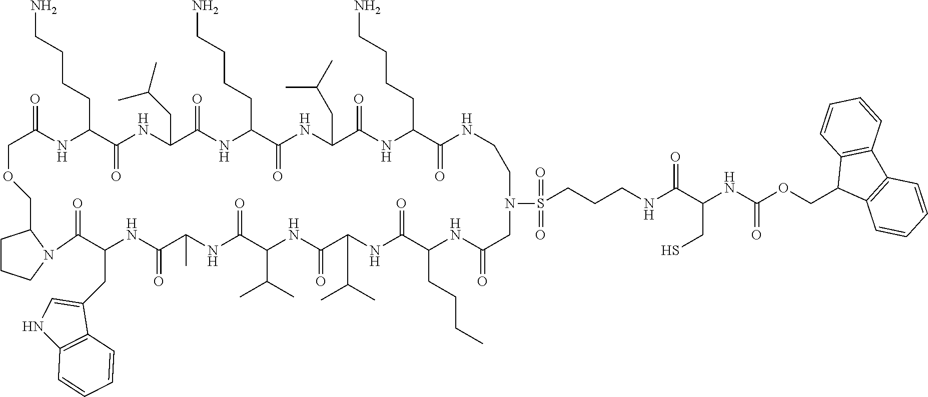 [SMILES: CCCCC1NC(=O)CN(S(=O)(=O)CCCNC(=O)C(CS)NC(=O)OCC2c3ccccc3-c3ccccc32)CCNC(=O)C(CCCCN)NC(=O)C(CC(C)C)NC(=O)C(CCCCN)NC(=O)C(CC(C)C)NC(=O)C(CCCCN)NC(=O)COCC2CCCN2C(=O)C(Cc2c[nH]c3ccccc23)NC(=O)C(C)NC(=O)C(C(C)C)NC(=O)C(C(C)C)NC1=O]
or a pharmaceutically acceptable salt, tautomer, or stereoisomer thereof.

74. The compound of claim 71, wherein the compound has the following structure: 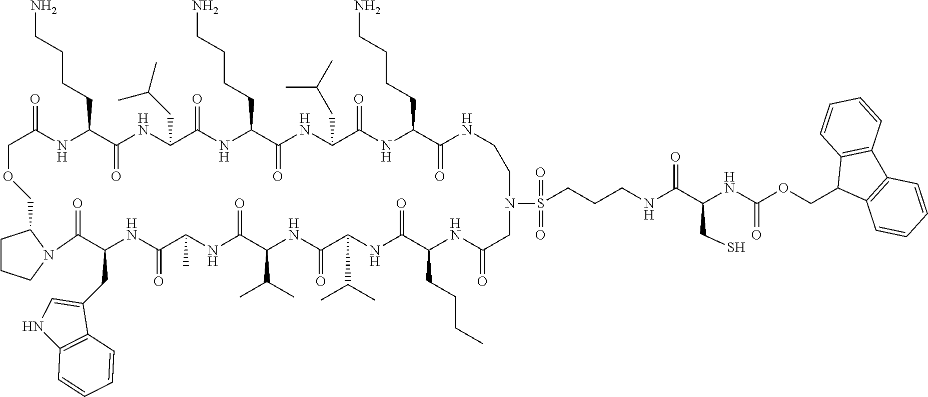 [SMILES: CCCC[C@@H]1NC(=O)CN(S(=O)(=O)CCCNC(=O)[C@H](CS)NC(=O)OCC2c3ccccc3-c3ccccc32)CCNC(=O)[C@H](CCCCN)NC(=O)[C@H](CC(C)C)NC(=O)[C@H](CCCCN)NC(=O)[C@H](CC(C)C)NC(=O)[C@H](CCCCN)NC(=O)COC[C@H]2CCCN2C(=O)[C@H](Cc2c[nH]c3ccccc23)NC(=O)[C@H](C)NC(=O)[C@H](C(C)C)NC(=O)[C@H](C(C)C)NC1=O]
or a pharmaceutically acceptable salt or tautomer thereof.
